SEQUENCE LISTING (9 sequences)

SEQ ID NO: 1 - protein, 461 aa, Homo sapiens [sequence omitted]

SEQ ID NO: 2 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 5 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLRFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 6 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLASTKFTIYNNMFCAGFHEGGRDSCQGDSGGPHVTEVEGTSFLTGIISWGEECAMKGKYGIYTKVSRYVNWIKEKTKLT

SEQ ID NO: 7 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 8 - DNA, 1383 bp, Artificial Sequence
  Synthetic construct
atgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgaggagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacc

SEQ ID NO: 9 - DNA, 334 bp, Homo sapiens
ccctaaaatgggcaaacattgcaagcagcaaacagcaaacacacagccctccctgcctgctgaccttggagctggggcagaggtcagacacctctctgggcccatgccacctccaactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

(19) United States
(12) Patent Application Publication
(10) Pub. No.: US 2021/0395714 A1
(43) Pub. Date: Dec. 23, 2021

(54) MODIFIED FACTOR IX POLYPEPTIDES

(71) Applicant: UCL BUSINESS LTD, London (GB)

(72) Inventors: Amit NATHWANI, London (GB); Nishil PATEL, London (GB); Keith GOMEZ, London (GB)

(21) Appl. No.: 17/289,598

(22) PCT Filed: Oct. 30, 2019
(86) PCT No.: PCT/GB2019/053071

§ 371 Date: Apr. 28, 2021

(30) Foreign Application Priority Data
Oct. 31, 2018 (GB) 1817810.3

(51) Int. Cl. C12N 9/64; A61K 38/48; C12N 7/00; C12N 15/86

(52) U.S. Cl. CPC C12N 9/644; A61K 38/4846; C12N 7/00; C12N 2830/50; C12Y 304/21022; C12N 2750/14142; C12N 15/86

(57) ABSTRACT

The present invention relates to modified Factor IX polypeptides comprising a mutation at a position corresponding to position 347 of wild type immature (precursor) Factor IX, polynucleotides encoding the polypeptides, and treatments utilising the polypeptides or polynucleotides.

### FIELD OF THE INVENTION

The present invention relates to modified Factor IX polypeptides comprising a mutation at a position corresponding to position 347 of wild type immature (precursor) Factor IX, polynucleotides encoding the polypeptides, and treatments utilising the polypeptides or polynucleotides.

### BACKGROUND TO THE INVENTION

Haemophilia B, an X-linked life-threatening bleeding disorder affects 1:30,000 males. Current treatment involves frequent intravenous injections (2-3 times per week) of Factor IX (FIX) protein. This treatment is highly effective at arresting bleeding but it is not curative and is extremely expensive (£150,000/patient/year), thus making it unaffordable by the majority of haemophilia B patients in the world. Gene therapy for haemophilia B offers the potential for a cure through persistent, endogenous production of Factor IX following the transfer of a functioning copy of the Factor IX gene to an affected patient. However, levels of Factor IX expressed from the Factor IX gene in patients receiving a gene therapy are generally not as high as in healthy patients. Thus, mechanisms to improve the efficacy of vectors carrying a Factor IX transgene, i.e. to increase transduction rates, increase Factor IX expression and/or increase the activity of the expressed Factor IX are desirable.

The present application relates to a modified Factor IX polypeptide comprising a mutation at a position corresponding to position 347 of wild type immature Factor IX. The present inventors surprisingly found that mutations at a position corresponding to position 347 of the wild type immature Factor IX can provide a hyper-active Factor IX polypeptide. Such a hyper-active Factor IX polypeptide can be useful in a variety of applications. For example, polynucleotides encoding such a hyper-active Factor IX polypeptide are particularly useful when administered as part of the gene therapy.

### SUMMARY OF THE INVENTION

The present application demonstrates that a mutation at position 347 of wild type immature Factor IX leads to a hyper-active Factor IX polypeptide. Furthermore, the present application demonstrates that mutations at positions corresponding to both position 347 and position 384 of wild type immature Factor IX can lead to an even more active modified Factor IX polypeptide.

Position 347 of wild type immature (i.e. precursor, zymogen form) Factor IX is part of an exposed loop (Y341-F348; numbering based on immature form) on the side of the Factor IX catalytic domain, which interacts with Factor VIIIa. Regions within the catalytic domain (residues 226-461; numbering based on immature form) may be involved in Factor VIIIa binding. Gain-of-function mutations have not been described in the Y341-F348 loop, but in contrast, variations in the 347-349 region are known to lead to reduced activity and a bleeding phenotype. Replacement of lysine 347 with arginine has been surprisingly shown to provide a significantly increased Factor IX activity (i.e. a “gain-of-function”) compared to wild type (i.e. unmutated) Factor IX. Without wishing to be bound by theory, it is thought that is due to better binding Factor VIIIa.

Region 379-385 of Factor IX is also associated with binding of Factor VIIIa. Thus, one might assume that gain-of-function mutations in this region might interact unfavourably with a mutation at position 347, or that one mutation may be “redundant” and effectively inconsequential in the presence of the other. However, the present application demonstrates that mutations at positions 347 and 384 interact favourably with one another to provide a Factor IX polypeptide that is even more active than a polypeptide mutated at position 347 alone or a polypeptide mutated at position 384 alone. In particular, modified Factor IX polypeptides having K347R combined with R384A or R384L mutations are particularly active.

Accordingly, in the first aspect of the invention, there is provided a modified Factor IX polypeptide comprising a mutation at a position corresponding to position 347 of SEQ ID NO: 1.

In a second aspect of the invention, there is provided a polynucleotide comprising a Factor IX nucleotide sequence that encodes the modified Factor IX polypeptide of the invention.

In a third aspect of the invention, there is provided a viral particle comprising a recombinant genome comprising the polynucleotide of the invention.

In a fourth embodiment there is provided a composition comprising the modified Factor IX polypeptide, polynucleotide or viral particle of the invention and a pharmaceutically acceptable excipient.

In a fifth embodiment, there is provided a method of treatment comprising administering an effective amount of the modified Factor IX polypeptide, polynucleotide, viral particle or composition of the invention to a patient.

In a sixth embodiment, there is provided a use of the modified Factor IX polypeptide, polynucleotide, viral particle or composition of the invention in the manufacture of a medicament for use in a method of treatment.

### DESCRIPTION OF THE FIGURES

FIG. 1—Schematic of Factor IX structure. The numbers above the schematic represent amino acid positions in the complete Factor IX polypeptide including the signal peptide and the pro-peptide region (SEQ ID NO: 1). The numbers below the schematic represent equivalent amino acid positions in mature Factor IX (which corresponds to SEQ ID NO: 2).

FIG. 2—Activity of Factor IX variants from Factor IX knock out mice transduced with r-hFIX-expressing AAV. (A) Chromogenic FIX activity levels (expressed as U/ml) in plasma from mice collected at 2-, 4-, 8-, and 13-weeks post injection with AAV vector encoding r-hFIX-WT (i.e. unmutated r-hFIX), r-hFIX-K347L, r-hFIX-K347R and r-hFIX-R384L. (B) Plasma FIX antigen levels (expressed as U/ml) from mice as described in (A). (C) Specific activity of FIX from mice as described in (A). The data represent the average ±1 standard deviation of four to five animals per group.

FIG. 3—Specific activity of FIX variants from stable cell lines. The antigen level of r-hFIX WT, K347R, R384L, K347R+R384L, R384A and K347R+R384A was measured in duplicate as described in material and methods. The specific activity was obtained by dividing the level of antigen over the level of activity. The data represent the average ±1 standard deviation of three independent experiments.

FIG. 4—Specific activity of purified FIX variants. Intrinsic one stage FIX clotting activity (APTT) (panel A) and chromogenic FIX activity (panel B) of r-hFIX-WT, K347R, R384L and K347R+R384L spiked into FIX deficient plasma at a concentration of 0.25 μg/ml. The data obtained by one-stage clotting assay or chromogenic assay (expressed as milli-Units/ml) have been divided by the protein concentration (μg/ml) to give specific activity (mU/μg). The data represent the average ±1 standard deviation of six independent experiments.

FIG. 5—Sequence Listing

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a modified Factor IX polypeptide comprising a mutation at a position corresponding to position 347” should be interpreted to mean that the polypeptide has a mutation at position 347, but the polypeptide may comprise further mutations. Similarly, the phrase “a polynucleotide comprising a Factor IX nucleotide sequence” refers to a polynucleotide that has a Factor IX nucleotide sequence, but the polynucleotide may contain additional nucleotides.

In some embodiments of the invention, the word “comprising” is replaced with the phrase “consisting of”. The term “consisting of” is intended to be limiting. For example, the phrase “a polynucleotide consisting of a Factor IX nucleotide sequence” should be understood to mean that the polynucleotide has a Factor IX nucleotide sequence and no additional nucleotides.

The terms “protein” and “polypeptide” are used interchangeably herein, and are intended to refer to a polymeric chain of amino acids of any length.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide or amino acid residues at each position are then compared. When a position in the first sequence is occupied by the same amino acid as the corresponding position in the second sequence, then the amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

For the purposes of the present invention, the term “fragment” refers to a contiguous portion of a sequence. For example, a fragment of SEQ ID NO: 1 of 50 amino acids refers to 50 contiguous nucleotides of SEQ ID NO: 1.

### A Modified Factor IX Polypeptide

In one aspect, the present invention provides a modified Factor IX (FIX) polypeptide comprising a mutation at a position corresponding to position 347 of SEQ ID NO: 1.

The term “modified Factor IX polypeptide” refers to a polypeptide that is homologous to Factor IX but does not have the sequence of wild type Factor IX, i.e. does not have a sequence that is identical to SEQ ID NO: 1, SEQ ID NO: 2, SEQ ID NO: 3, or SEQ ID NO: 4. For example, a polypeptide comprising a sequence of SEQ ID NO: 1, but comprising a mutation at a position corresponding to position 347 is a modified Factor IX polypeptide.

Wild type Factor IX is a serine protease, which forms part of the coagulation cascade. Lack of or mutated Factor IX can lead to reduced blood clotting and the disease haemophilia B. A typical wild type Factor IX polypeptide is present in SEQ ID NO: 1 (sometimes referred to as Factor IX Malmo B) or SEQ ID NO: 2 (mature (active form) Factor IX Malmo B). An alternative wild type Factor IX polypeptide differs from that encoded by SEQ ID NO: 1 at a position corresponding to position 194 of SEQ ID NO: 1. For example, position 194 may be a threonine amino acid (“Malmo A”) instead of alanine (as in SEQ ID NO: 3).

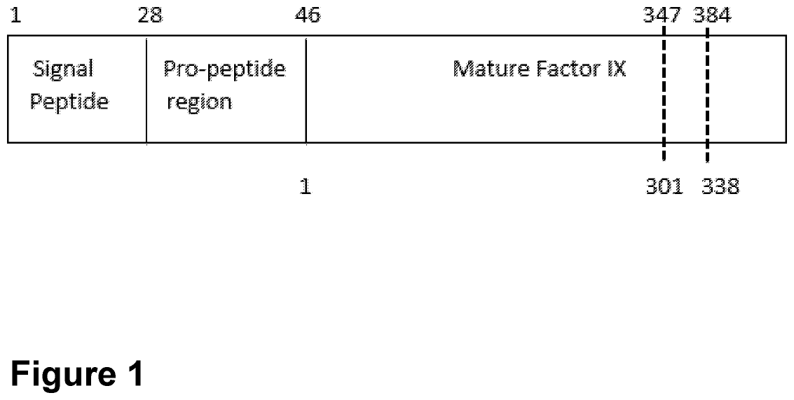

Factor IX (e.g. a Factor IX of SEQ ID NO: 1) as initially expressed as a precursor “immature” form, comprising a hydrophobic signal peptide (amino acids 1-28 of SEQ ID NO: 1 or SEQ ID NO: 3), a pro-peptide region (amino acids 29-46 of SEQ ID NO: 1 or SEQ ID NO: 3) and a mature polypeptide region, as set out in FIG. 1. The mature (zymogen) form of Factor IX lacks the hydrophobic signal peptide and the pro-peptide region, and is represented by SEQ ID NO: 2 or SEQ ID NO: 4. The term “mature Factor IX” refers to a Factor IX polypeptide that does not comprise the hydrophobic signal peptide or the pro-peptide region, such as SEQ ID NO. 2 or SEQ ID NO: 4.

During clotting the single-chain zymogen form is cleaved by Factor XIa or Factor VIIa to produce an active two-chain form (Factor IXa), with the two chains linked by a disulphide bridge. The activated form can catalyse the hydrolysis of an arginine-isoleucine bond in Factor X to form Factor Xa. Wild type Factor IX is inhibited by thrombin. The wild type Factor IX protein has four protein domains, a Gla domain, two tandem copies of the EGF domain and a C-terminal trypsin-like peptidase domain which is responsible for catalytic cleavage.

The term “Factor IX polypeptide” refers to a polypeptide that is homologous to the single-chain zymogen form of Factor IX, the activated two-chain form and variants thereof, and may refer to a mature Factor IX polypeptide or a Factor IX polypeptide comprising the pro-peptide region and/or the signal peptide region.

### A Mutation at a Position Corresponding to Position 347 of SEQ ID NO: 1

The modified Factor IX polypeptide of the invention comprises a mutation at a position corresponding to position 347 of SEQ ID NO: 1. As discussed above, SEQ ID NO: 1 is a sequence of wild type Factor IX, and SEQ ID NO: 2 is a sequence of a mature form of wild type Factor IX (lacking the first 46 amino acids that correspond to the pro-peptide and signal peptide regions). Position 347 of SEQ ID NO: 1 corresponds to position 301 of SEQ ID NO: 2.

In order to determine whether a Factor IX polypeptide has a mutation at a position corresponding to position 347 of SEQ ID NO: 1, the user may align the Factor IX polypeptide with SEQ ID NO: 1 using a suitable algorithm such as that of Needleman and Wunsch described above, and determine the nature of the amino acid that is present at the position that aligns with position 347 of SEQ ID NO: 1. Position 347 of SEQ ID NO: 1 is a lysine residue. Thus, if the amino acid that is present at the position that aligns with position 347 of SEQ ID NO: 1 is not a lysine residue, then the Factor IX polypeptide comprises a mutation at a position corresponding to position 347 of SEQ ID NO: 1.

The mutation at a position corresponding to position 347 of SEQ ID NO: 1 is preferably a gain-of-function mutation. A gain-of-function mutation is a mutation that increases the activity of the modified Factor IX polypeptide. As discussed above, Factor IX polypeptide converts Factor X to Factor Xa, and so a mutation at a position corresponding to position 347 of SEQ ID NO: 1 is a gain-of-function mutation if it increases the rate at which the modified Factor IX polypeptide converts Factor X to Factor Xa. For example, replacement of lysine 347 with an arginine amino acid increases the specific activity of a Factor IX polypeptide, as demonstrated in Examples 1-3.

It is within the ability of the person skilled in the art to determine whether a mutation at a position corresponding to position 347 of SEQ ID NO: 1 is a gain-of-function mutation. One merely needs to compare a modified Factor IX polypeptide comprising the mutation with an equivalent polypeptide lacking a mutation at a position corresponding to position 347, and see whether the modified Factor IX polypeptide comprising the mutation has higher activity compared to the polypeptide lacking a mutation at a position corresponding to position 347 of SEQ ID NO: 1. If the modified Factor IX polypeptide comprising a mutation at a position corresponding to position 347 of SEQ ID NO: 1 does have a higher activity compared to the polypeptide lacking a mutation at a position corresponding to position 347 of SEQ ID NO: 1, then the mutation at a position corresponding to position 347 of SEQ ID NO: 1 is a gain-of-function mutation.

“An equivalent polypeptide lacking a mutation at a position corresponding to position 347” is a modified Factor IX polypeptide that has an identical sequence except for the mutation at a position corresponding to position 347 of SEQ ID NO: 1, i.e. the polypeptide has an identical sequence except that the amino acid at a position corresponding to position 347 is a lysine residue.

There are various methods for determining the activity of a Factor IX polypeptide, such as a Factor IX polypeptide comprising a mutation at a position corresponding to position 347 and an equivalent polypeptide lacking a mutation at a position corresponding to position 347. These methods include a chromogenic assay, a clotting assay or a tail clip assay. Suitable chromogenic assays and clotting assays are described in Examples 1-3.

For example, a suitable chromogenic assay is as follows. The Factor IX polypeptide is mixed with human Factor X, human Factor VIII and calcium. Factor IXa activity is subsequently initiated by addition of thrombin, Factor XIa and phospholipids. Under these conditions, the Factor XIa activates the Factor IX polypeptide to form a Factor IXa polypeptide, and thrombin activates the FVIII polypeptide to form a FVIIIa polypeptide, which enables formation of the intrinsic Xase (FIXa/FVIIIa) complex that catalyses the conversion of Factor X to Factor Xa. The activity of the Factor Xa polypeptide can catalyse cleavage of a chromogenic substrate (e.g. SXa-11) to produce pNA. The level of pNA generated can be measured by determining absorbance at 405 nm, and this is proportional to the amount of the Factor Xa polypeptide generated by FIXa in the sample, which is proportional to the activity of FIXa in the sample.

Similarly, a suitable clotting assay (a one-stage clotting assay) is as follows. Since Factor IX is part of the clotting cascade, a Factor IX polypeptide that has increased activity will catalyse blood clotting more quickly that a Factor IX polypeptide that has a lower activity.

For example, a suitable clotting assay is as follows. The Factor IX polypeptide is mixed with platelet poor plasma, and incubated at 37° C. Then phospholipid and a contact activation pathway activator such as Kaolin or SynthaSIL APTT reagent are added. Calcium is then added, and the user measures the time taken for clotting to occur. The clot formation can be assessed using spectrophotometry or by a magnetic steel ball method. For example, clot formation may be deemed to have occurred with the optical density of the mixture exceeds a threshold or when the mixture has congealed sufficiently to alter the movement of the magnetic ball, which is detected by the sensor.

A suitable tail clip assay may involve administering, Factor IX polypeptide, or polynucleotide in the context of a gene therapy, to mice such as knock-out mice deficient in blood clotting. The tails of the mice are then clipped, and the time taken for the cut in the tails to clot is measured. The duration of bleeding provides a relative measure of the activity of the administered Factor IX, for example gain-of-function-containing vs wild type Factor IX.

In preferred embodiments, the Factor IX polypeptide is purified, and the specific activity is measured by a clotting assay or a chromogenic assay carried out on the purified Factor IX polypeptide. In some embodiments, the specific activity of the Factor IX polypeptide is measured by generating an AAV vector comprising a transgene encoding the Factor IX polypeptide, injecting mice with the AAV vector, and detecting the specific activity in plasma from the mice using a chromogenic assay. In some embodiments, the specific activity of the Factor IX polypeptide is measured by providing cells stably expressing a polynucleotide encoding the Factor IX polypeptide, harvesting Factor IX polypeptide from the cells and/or culture medium, and measuring the specific activity of the Factor IX polypeptide using a chromogenic assay.

For the purposes of the present application, the term “specific activity” refers to the activity (e.g. clotting activity or intrinsic Xase activity) per unit (e.g. per μg, or per antigen level as % of level in normal human plasma) of Factor IX polypeptide such that the activity is ‘normalised’ to take account of the amount or concentration of Factor IX polypeptide in the sample. (Note that typically, pooled healthy human plasma has a Factor IX concentration of 5 μg/ml.) This can be done by measuring the concentration of the Factor IX polypeptide in the sample using a standard ELISA assay, such as the assay described in Examples 1 and 2, and dividing the activity by the Factor IX concentration.

For example, an antibody that binds to the Factor IX polypeptide could be bound to a plate. The sample, comprising the Factor IX polypeptide at unknown concentration, could be passed over the plate. A second detection antibody that binds to the Factor IX polypeptide could be applied to the plate, and any excess washed off. The detection antibody that remains (i.e. is not washed off) will be bound to the Factor IX polypeptide. The detection antibody could be linked to an enzyme such as horse radish peroxidase. The level of detection antibody that binds to the Factor IX polypeptide on the plate could be measured by measuring the amount of the detection antibody. For example, if the detection antibody is linked to horse radish peroxidase, the horse radish peroxidase can catalyse the production of a blue reaction product from a substrate such as TMB (3,3′,5,5′-tetramethylbenzidine), and the level of the blue product can be detected by absorbance at 450 nm. The level of the blue product is proportional to the amount of detection antibody that remained after the washing step, which is proportional to the amount of the Factor IX polypeptide in the sample. Alternatively, for example when using purified protein, the amount or concentration of Factor IX polypeptide may be determined spectrophotometrically.

Optionally, the modified Factor IX polypeptide of the invention has a higher activity compared to an equivalent polypeptide lacking a mutation at a position corresponding to position 347 of SEQ ID NO: 1.

Optionally, the modified Factor IX polypeptide has a higher activity compared to a polypeptide of SEQ ID NO: 1, SEQ ID NO: 2, SEQ ID NO: 3 and/or SEQ ID NO: 4. Optionally, the modified Factor IX polypeptide has a higher activity compared to a polypeptide of SEQ ID NO: 1 or SEQ ID NO: 2. Optionally, the modified Factor IX polypeptide has a higher activity compared to a polypeptide of SEQ ID NO: 1, a polypeptide of SEQ ID NO: 2, a polypeptide of SEQ ID NO: 3 and a polypeptide of SEQ ID NO: 4. The activity of the modified Factor IX polypeptide and the polypeptide of SEQ ID NO: 1, SEQ ID NO: 2, SEQ ID NO: 3 and/or SEQ ID NO: 4 may be determined using the chromogenic assay, clotting assay, or tail clip assay as described above.

Optionally, the modified Factor IX polypeptide has a higher specific activity compared to an equivalent polypeptide lacking a mutation at a position corresponding to position 347 of SEQ ID NO: 1. Optionally, the modified Factor IX polypeptide has a higher specific activity compared to a polypeptide of SEQ ID NO: 1, SEQ ID NO: 2, SEQ ID NO: 3 and/or SEQ ID NO: 4. Optionally, the modified Factor IX polypeptide has a higher specific activity compared to a polypeptide of SEQ ID NO: 1 or SEQ ID NO: 2. Optionally, the modified Factor IX polypeptide has a higher specific activity compared to a polypeptide of SEQ ID NO: 1, SEQ ID NO: 2, SEQ ID NO: 3 and SEQ ID NO: 4.

Optionally, the specific activity is measured using a chromogenic assay, i.e. a chromogenic assay is used to determine the activity of the modified Factor IX polypeptide and, if necessary, the activity is normalised using an ELISA test such as described above. A suitable chromogenic assay is described above. Optionally, the modified Factor IX polypeptide has an at least 1.2 fold, at least 1.5 fold, at least 1.6 fold, at least 1.7 fold, or at least 1.8 fold higher specific activity than an equivalent polypeptide lacking a mutation at a position corresponding to position 347 of SEQ ID NO: 1, or compared to a polypeptide of SEQ ID NO: 1, SEQ ID NO: 2, SEQ ID NO: 3, or SEQ ID NO: 4. Optionally, the modified Factor IX polypeptide has an at least 1.2 fold, at least 1.5 fold, at least 1.6 fold, at least 1.7 fold, or at least 1.8 fold higher specific activity than a polypeptide of SEQ ID NO: 1 or SEQ ID NO: 2. Optionally, the modified Factor IX polypeptide has an at least 1.2 fold, at least 1.5 fold, at least 1.6 fold, at least 1.7 fold, or at least 1.8 fold higher specific activity than an equivalent polypeptide lacking a mutation at a position corresponding to position 347 of SEQ ID NO: 1. Optionally, the modified Factor IX polypeptide has an at least 1.2 fold, at least 1.5 fold, at least 1.6 fold, at least 1.7 fold, or at least 1.8 fold higher specific activity than a polypeptide of SEQ ID NO: 1, a polypeptide of SEQ ID NO: 2, a polypeptide of SEQ ID NO: 3 and a polypeptide of SEQ ID NO: 4.

Optionally, the specific activity is measured using a clotting assay i.e. a clotting assay, such as a one-stage clotting assay, is used to determine the activity of the modified Factor IX polypeptide and the activity is normalised using the ELISA test described above. A suitable clotting assay is described above. Optionally, the modified Factor IX polypeptide has at least 1.2 fold, at least 1.5 fold, at least 1.6 fold, at least 1.7 fold, or at least 1.8 fold higher specific activity compared to an equivalent polypeptide lacking a mutation at a position corresponding to position 347 of SEQ ID NO: 1, or compared to a polypeptide of SEQ ID NO: 1, SEQ ID NO: 2, SEQ ID NO: 3 or SEQ ID NO: 4. Optionally, the modified Factor IX polypeptide has at least 1.2 fold, at least 1.5 fold, at least 1.6 fold, at least 1.7 fold, or at least 1.8 fold higher specific activity compared to a polypeptide of SEQ ID NO: 1 or SEQ ID NO: 2. Optionally, the modified Factor IX polypeptide has at least 1.2 fold, at least 1.5 fold, at least 1.6 fold, at least 1.7 fold, or at least 1.8 fold higher specific activity compared to an equivalent polypeptide lacking a mutation at a position corresponding to position 347 of SEQ ID NO: 1. Optionally, the modified Factor IX polypeptide has at least 1.2 fold, at least 1.5 fold, at least 1.6 fold, at least 1.7 fold, or at least 1.8 fold higher specific activity compared to a polypeptide of SEQ ID NO: 1, a polypeptide of SEQ ID NO: 2, a polypeptide of SEQ ID NO: 3 and a polypeptide of SEQ ID NO: 4.

The mutation at a position corresponding to position 347 of SEQ ID NO: 1 may be a mutation to a large positively-polarised amino acid. Large positively-polarised amino acids include arginine, histidine, glutamine, asparagine, and tryptophan. The mutation at a position corresponding to position 347 of SEQ ID NO: 1 may be a mutation to an amino acid residue selected from the group consisting of arginine, histidine, and glutamine. Optionally, the mutation at a position corresponding to position 347 of SEQ ID NO: 1 is a mutation to arginine.

### A Mutation at a Position Corresponding to Position 384 of SEQ ID NO: 1

The modified Factor IX polypeptide of the invention may comprise additional mutations. Preferably, any additional mutations are gain-of-function mutations. For example, the modified Factor IX polypeptide of the invention may comprise a mutation at a position corresponding to position 384 of SEQ ID NO: 1.

In order to determine whether a Factor IX polypeptide has a mutation at a position corresponding to position 384 of SEQ ID NO: 1, the user may align the Factor IX polypeptide with SEQ ID NO: 1 using a suitable algorithm such as that of Needleman and Wunsch described above, and determine the nature of the amino acid that is present at the position that aligns with position 384 of SEQ ID NO: 1. Position 384 of SEQ ID NO: 1 is an arginine residue. Thus, if the amino acid that is present at the position that aligns with position 384 of SEQ ID NO: 1 is not an arginine residue, then the Factor IX polypeptide comprises a mutation at a position corresponding to position 384 of SEQ ID NO: 1.

The mutation at a position corresponding to position 384 of SEQ ID NO: 1 is preferably a gain-of-function mutation. For example, replacement of arginine 384 with a leucine or alanine amino acid leads to an increase in the specific activity of a Factor IX polypeptide, as demonstrated in Examples 1-3. Similarly, replacement of arginine 384 with glutamine or aspartate has been reported to lead to an increase in the specific activity of a Factor IX polypeptide.

It is within the ability of the person skilled in the art to determine whether a mutation at a position corresponding to position 384 of SEQ ID NO: 1 is a gain-of-function mutation.

One merely needs to compare a Factor IX polypeptide comprising the mutation with an equivalent polypeptide lacking a mutation at a position corresponding to position 384 and see whether the Factor IX polypeptide comprising the mutation has higher activity compared to the polypeptide lacking a mutation at a position corresponding to position 384. If the Factor IX polypeptide comprising the mutation at a position corresponding to position 384 of SEQ ID NO: 1 does have a higher activity compared to the polypeptide lacking a mutation at a position corresponding to position 384, then the mutation is a gain-of-function mutation.

“An equivalent polypeptide lacking a mutation at a position corresponding to position 384” is a Factor IX polypeptide that has an identical sequence except for the mutation at a position corresponding to position 384, i.e. the polypeptide has an identical sequence except that the amino acid at a position corresponding to position 384 is an arginine residue.

Suitable assays for determining the specific activity of a Factor IX polypeptide are described above, under the heading “A mutation at a position corresponding to position 347 of SEQ ID NO: 1”. Suitable assays include chromogenic assays, clotting assays and tail clip assays, such as those described above.

Optionally, the mutation at a position corresponding to position 384 of SEQ ID NO: 1 is a mutation to a small non-polar amino acid. Small non-polar amino acids include alanine, valine, leucine and isoleucine. Optionally, the mutation at a position corresponding to position 384 of SEQ ID NO: 1 is a mutation to an amino acid selected from the group consisting of alanine, valine, leucine and isoleucine. Optionally, the mutation at a position corresponding to position 384 of SEQ ID NO: 1 is a mutation to alanine. Optionally, the mutation at a position corresponding to position 384 of SEQ ID NO: 1 is a mutation to leucine. The mutation at a position corresponding to position 384 of SEQ ID NO: 1 may be a mutation to glutamine or aspartate.

The modified Factor IX polypeptide may have mutations at both a position corresponding to position 347 and a position corresponding to position 384 of SEQ ID NO: 1. For example, the modified Factor IX polypeptide may have a mutation to a large positively charged amino acid at a position corresponding to position 347 of SEQ ID NO: 1, and a mutation to a small non-polar amino acid at a position corresponding to position 384 of SEQ ID NO: 1. Similarly, the modified Factor IX polypeptide may have a mutation to an amino acid residue selected from the group consisting of arginine, histidine, and glutamine at a position corresponding to position 347 of SEQ ID NO: 1, and a mutation to an amino acid selected from the group consisting of glutamine, aspartate, alanine, valine, leucine and isoleucine at a position corresponding to position 384 of SEQ ID NO: 1. The modified Factor IX polypeptide may have a mutation to arginine at a position corresponding to position 347 of SEQ ID NO: 1, and a mutation to a small non-polar amino acid at a position corresponding to position 384 of SEQ ID NO: 1. The modified Factor IX polypeptide may have a mutation to arginine at a position corresponding to position 347 of SEQ ID NO: 1, and a mutation to an amino acid selected from the group consisting of alanine, valine, leucine and isoleucine at a position corresponding to position 384 of SEQ ID NO: 1. The modified Factor IX polypeptide may have a mutation to arginine at a position corresponding to position 347 of SEQ ID NO: 1, and a mutation to alanine or leucine at a position corresponding to position 384 of SEQ ID NO: 1.

The modified Factor IX polypeptide may have a higher specific activity compared to an equivalent polypeptide lacking a mutation at a position corresponding to position 347 of SEQ ID NO: 1 and lacking a mutation at a position corresponding to position 384 of SEQ

ID NO: 1. “An equivalent polypeptide lacking a mutation at a position corresponding to position 347 of SEQ ID NO: 1 and a mutation at a position corresponding to position 384 of SEQ ID NO: 1” is a modified Factor IX polypeptide that has an identical sequence except for the mutations at positions corresponding to position 347 and 384, i.e. the polypeptide has an identical sequence except that the amino acid at a position corresponding to position 347 is a lysine residue and the amino acid at a position corresponding to position 384 is an arginine residue.

The present application demonstrates that a Factor IX polypeptide having arginine at a position corresponding to position 347 of SEQ ID NO: 1, and leucine or alanine at a position corresponding to position 384 of SEQ ID NO: 1 has higher activity compared to an otherwise identical (equivalent) Factor IX polypeptide having only a single mutation to arginine at a position corresponding to position 347 of SEQ ID NO: 1, as well as higher activity compared to an otherwise identical (equivalent) Factor IX polypeptide having only a single mutation to either leucine or alanine at a position corresponding to position 384 of SEQ ID NO: 1.

Optionally, the modified Factor IX polypeptide has a higher specific activity compared to a polypeptide of SEQ ID NO: 5. Optionally, the modified Factor IX polypeptide has an at least 2.5 fold, at least 3 fold, at least 3.3 fold, at least 4 fold, at least 4.5 fold, at least 5 fold, at least 5.5 fold, or at least 6 fold higher specific activity compared to a polypeptide of SEQ ID NO: 5. Optionally, the modified Factor IX polypeptide has a higher specific activity compared to a polypeptide of SEQ ID NO: 6. Optionally, the modified Factor IX polypeptide has an at least 1.1 fold, at least 1.2 fold, at least 2.0 fold, or at least 2.2 fold higher specific activity compared to a polypeptide of SEQ ID NO: 6. Optionally, the modified Factor IX polypeptide has a higher specific activity compared to a polypeptide of SEQ ID NO: 7. Optionally, the modified Factor IX polypeptide has an at least 1.1 fold, at least 1.15 fold, at least 1.3 fold, at least 1.5 fold, or at least 1.6 fold higher specific activity compared to a polypeptide of SEQ ID NO: 7.

The modified Factor IX polypeptide may have a higher specific activity compared to a polypeptide of SEQ ID NO: 5, and a higher specific activity compared to a polypeptide of SEQ ID NO: 6. The modified Factor IX polypeptide may have a higher specific activity compared to a polypeptide of SEQ ID NO: 5, and a higher specific activity compared to a polypeptide of SEQ ID NO: 7. The modified Factor IX polypeptide may have a higher specific activity compared to a polypeptide of SEQ ID NO: 5, a higher specific activity compared to a polypeptide of SEQ ID NO: 6, and a higher specific activity compared to a polypeptide of SEQ ID NO: 7. The modified Factor IX polypeptide may have an at least 5 fold higher specific activity compared to a polypeptide of SEQ ID NO: 5, an at least 2.0 fold higher specific activity compared to a polypeptide of SEQ ID NO: 6, and an at least 1.5 fold higher specific activity compared to a polypeptide of SEQ ID NO: 7.

Suitable assays for determining the specific activity of a Factor IX polypeptide are described above under the heading “A mutation at a position corresponding to position 347 of SEQ ID NO: 1”. For example, the specific activity may be measured using a chromogenic assay, a clotting assay or a tail clip assay such as described above.

### Homology to SEQ ID NO: 1 or SEQ ID NO: 2

The modified Factor IX polypeptide may comprise a polypeptide sequence at least 80%, at least 90%, at least 95%, at least 98%, or at least 99% identical to a fragment of SEQ ID NO: 1 or SEQ ID NO: 2 of at least 200, at least 250, at least 300, between 200 and 415, between 250 and 415, or between 300 and 415 amino acids.

Preferably, the modified Factor IX polypeptide is functional. A functional Factor IX polypeptide is one which carries out hydrolysis of an arginine-isoleucine bond in Factor X to form Factor Xa. It is within the abilities of the skilled person to determine whether a Factor IX polypeptide is functional. One merely needs to determine whether the modified Factor IX polypeptide is active in a chromogenic assay, clotting assay or tail clip assay as described under the heading “A mutation at a position corresponding to position 347 of SEQ ID NO: 1”. Preferably, a functional modified Factor IX polypeptide has at least 50%, at least 60%, at least 70%, at least 80%, at least 90%, or at least 100% of the specific activity of a polypeptide of SEQ ID NO: 1. As described above, in some embodiments, the modified Factor IX polypeptide has higher activity compared to a polypeptide of SEQ ID NO: 1 as the modified Factor IX polypeptide comprises a mutation at a position corresponding to position 347 of SEQ ID NO: 1, and such mutations may be gain-of-function mutations.

Optionally, the modified Factor IX polypeptide comprises a polypeptide sequence at least 80%, at least 90%, at least 95%, at least 98%, or at least 99% identical to SEQ ID NO: 1 or SEQ ID NO: 2. Optionally, the modified Factor IX polypeptide comprises a polypeptide sequence at least 98% identical to a fragment of between 300 and 415 amino acids of SEQ ID NO: 1. Optionally, the modified Factor IX polypeptide comprises a polypeptide sequence at least 98% identical to a fragment of between 300 and 415 amino acids of SEQ ID NO: 2. Optionally, the modified Factor IX polypeptide comprises a polypeptide sequence at least 98%, at least 98.5%, at least 99%, or at least 99.5% identical to SEQ ID NO: 1. Optionally, the modified Factor IX polypeptide comprises a polypeptide sequence at least 98%, at least 98.5%, at least 99%, or at least 99.5% identical to SEQ ID NO: 2. Optionally, the modified Factor IX polypeptide comprises a polypeptide sequence that is identical to SEQ ID NO:1 or SEQ ID NO: 2, except that the Factor IX polypeptide comprises a mutation at a position corresponding to position 347 of SEQ ID NO: 1, i.e. the modified Factor IX polypeptide comprises a polypeptide sequence that is identical to SEQ ID NO: 1 or SEQ ID NO: 2, except that the Factor IX polypeptide has an amino acid that is not lysine at a position corresponding to position 347 of SEQ ID NO: 1.

Optionally, the modified Factor IX polypeptide comprises a polypeptide sequence that is identical to SEQ ID NO: 1 or SEQ ID NO: 2, except that the modified Factor IX polypeptide comprises a mutation at a position corresponding to position 347 of SEQ ID NO: 1 and a mutation at a position corresponding to position 384 of SEQ ID NO: 1, i.e. the modified Factor IX polypeptide comprises a polypeptide sequence that is identical to SEQ ID NO: 1 or SEQ ID NO: 2, except that the modified Factor IX polypeptide has an amino acid that is not lysine at a position corresponding to position 347 of SEQ ID NO: 1 and an amino acid that is not arginine at a position corresponding to position 384 of SEQ ID NO: 1.

### A Polynucleotide Comprising a Factor IX Nucleotide Sequence

In one aspect, the present invention provides a polynucleotide comprising a Factor IX nucleotide sequence that encodes a modified Factor IX polypeptide of the invention.

The term “polynucleotide” refers to a polymeric form of nucleotides of any length, deoxyribonucleotides, ribonucleotides, or analogs thereof. For example, the polynucleotide may comprise DNA (deoxyribonucleotides) or RNA (ribonucleotides). The polynucleotide may consist of DNA. The polynucleotide may be mRNA. Since the polynucleotide may comprise RNA or DNA, all references to T (thymine) nucleotides may be replaced with U (uracil).

Optionally, the Factor IX nucleotide sequence is at least 80%, at least 90%, at least 95%, at least 98%, or at least 99% identical to a fragment of at least 900, at least 1000, at least 1300, or at least 1380 nucleotides of SEQ ID NO: 8. Optionally, the Factor IX nucleotide sequence is at least 80%, at least 90%, at least 95%, at least 98%, or at least 99% identical to SEQ ID NO: 8. Optionally, the Factor IX nucleotide sequence is at least 98% identical to a fragment of at least 1300 nucleotides of SEQ ID NO: 8. Optionally, the Factor IX nucleotide sequence is at least 98%, at least 98.5%, at least 99%, or at least 99.5% identical to SEQ ID NO: 8. Optionally, the Factor IX nucleotide sequence comprises a polynucleotide sequence that is identical to SEQ ID NO: 8, except that the Factor IX nucleotide sequence comprises a codon that encodes an amino acid at a position corresponding to position 347 of SEQ ID NO: 1 that is not lysine, and a codon that encodes an amino acid at a position corresponding to position 384 of SEQ ID NO: 1 that is not arginine. Optionally, the Factor IX nucleotide sequence comprises a polynucleotide sequence that is identical to SEQ ID NO: 8, except that the Factor IX nucleotide sequence comprises a codon that encodes an amino acid at a position corresponding to position 347 of SEQ ID NO: 1 that is not lysine.

In order to determine whether a Factor IX nucleotide sequence comprises a codon that encodes an amino acid at a position corresponding to position 347 that does not encode lysine, the user may align the Factor IX nucleotide sequence with SEQ ID NO: 8 using a suitable algorithm such as that of Needleman and Wunsch described above, and determine the nature of the codon that is present at the position that aligns with codon 347 (nucleotides 1039-1041) of SEQ ID NO: 8. Codon 347 of SEQ ID NO: 8 encodes a lysine residue. Thus, if the codon that is present at the position that aligns with codon 347 of SEQ ID NO: 8 does not encode a lysine residue, then the Factor IX nucleotide sequence comprises a codon that encodes an amino acid at a position corresponding to position 347 that is not lysine.

Similarly, in order to determine whether a Factor IX nucleotide sequence comprises a codon that encodes an amino acid at a position corresponding to position 384 that does not encode arginine, the user may align the Factor IX nucleotide sequence with SEQ ID NO: 8 using a suitable algorithm such as that of Needleman and Wunsch described above, and determine the nature of the codon that is present at the position that aligns with codon 384 (nucleotides 1150-1152) of SEQ ID NO: 8. Codon 384 of SEQ ID NO: 8 encodes an arginine residue. Thus, if the codon that is present at the position that aligns with codon 384 of SEQ ID NO: 8 does not encode an arginine residue, then the Factor IX nucleotide sequence comprises a codon that encodes an amino acid at a position corresponding to position 384 that is not arginine.

Optionally, the Factor IX nucleotide sequence comprises a codon that encodes an amino acid at a position corresponding to position 347 of SEQ ID NO: 1, wherein the codon that encodes an amino acid at a position corresponding to position 347 of SEQ ID NO: 1 encodes a large positively-polarised amino acid. Optionally, the Factor IX nucleotide sequence comprises a codon that encodes an amino acid at a position corresponding to position 347 of SEQ ID NO: 1, wherein the codon that encodes an amino acid at a position corresponding to position 347 of SEQ ID NO: 1 encodes an amino acid selected from the group consisting of arginine, histidine and glutamine. Optionally, the Factor IX nucleotide sequence comprises a codon that encodes an amino acid at a position corresponding to position 347 of SEQ ID NO: 1, wherein the codon that encodes an amino acid at a position corresponding to position 347 of SEQ ID NO: 1 encodes arginine.

Arginine may be encoded by any of CGT, CGC, CGA, CGG, AGA or AGG codons. Thus, the codon that encodes an amino acid at a position corresponding to position 347 of SEQ ID NO: 1 may be CGT, CGC, CGA, CGG, AGA or AGG. Optionally, the codon that encodes an amino acid at a position corresponding to position 347 of SEQ ID NO: 1 is AGG.

Optionally, the Factor IX nucleotide sequence comprises a codon that encodes an amino acid at a position corresponding to position 384 of SEQ ID NO: 1, wherein the codon that encodes an amino acid at a position corresponding to position 384 of SEQ ID NO: 1 encodes a small non-polar amino acid. Optionally, the Factor IX nucleotide sequence comprises a codon that encodes an amino acid at a position corresponding to position 384 of SEQ ID NO: 1, wherein the codon that encodes an amino acid at a position corresponding to position 384 of SEQ ID NO: 1 encodes an amino acid selected from the group consisting of glutamine, aspartate, alanine, valine, leucine and isoleucine. Optionally, the Factor IX nucleotide sequence comprises a codon that encodes an amino acid at a position corresponding to position 384 of SEQ ID NO: 1, wherein the codon that encodes an amino acid at a position corresponding to position 384 of SEQ ID NO: 1 encodes an amino acid selected from the group consisting of alanine, valine, leucine and isoleucine. Optionally, the Factor IX nucleotide sequence comprises a codon that encodes an amino acid at a position corresponding to position 384 of SEQ ID NO: 1, wherein the codon that encodes an amino acid at a position corresponding to position 384 of SEQ ID NO: 1 encodes alanine. Optionally, the Factor IX nucleotide sequence comprises a codon that encodes an amino acid at a position corresponding to position 384 of SEQ ID NO: 1, wherein the codon that encodes an amino acid at a position corresponding to position 384 of SEQ ID NO: 1 encodes leucine.

Alanine may be encoded by any of GCT, GCC, GCA or GCG codons. Thus, the codon that encodes an amino acid at a position corresponding to position 384 of SEQ ID NO: 1 may be GCT, GCC, GCA or GCG. Optionally, the codon that encodes an amino acid at a position corresponding to position 384 of SEQ ID NO: 1 is GCC.

Leucine may be encoded by any of TTA, TTG, CTT, CTC, CTA or CTG codons. Thus, the codon that encodes an amino acid at a position corresponding to position 384 of SEQ ID NO: 1 may be TTA, TTG, CTT, CTC, CTA or CTG. Optionally, the codon that encodes an amino acid at a position corresponding to position 384 of SEQ ID NO: 1 is CTX, wherein X is any nucleotide. Optionally, the codon that encodes an amino acid at a position corresponding to position 384 of SEQ ID NO: 1 is CTC or CTG.

The Factor IX nucleotide sequence may comprise a codon encoding a large positively-polarised amino acid at a position corresponding to position 347 of SEQ ID NO: 1, and a codon encoding a small non-polar amino acid at a position corresponding to position 384 of SEQ ID NO: 1. The Factor IX nucleotide sequence may comprise a codon encoding arginine, histidine or glutamine at a position corresponding to position 347 of SEQ ID NO: 1, and a codon encoding a small non-polar amino acid at a position corresponding to position 384 of SEQ ID NO: 1. The Factor IX nucleotide sequence may comprise a codon encoding arginine at a position corresponding to position 347 of SEQ ID NO: 1, and a codon encoding a small non-polar amino acid at a position corresponding to position 384 of SEQ ID NO: 1.

The Factor IX nucleotide sequence may comprise a codon encoding a large positively-polarised amino acid at a position corresponding to position 347 of SEQ ID NO: 1, and a codon encoding alanine, leucine, isoleucine or valine at a position corresponding to position 384 of SEQ ID NO: 1. The Factor IX nucleotide sequence may comprise a codon encoding arginine, histidine or glutamine at a position corresponding to position 347 of SEQ ID NO: 1, and a codon encoding alanine, leucine, isoleucine or valine at a position corresponding to position 384 of SEQ ID NO: 1. The Factor IX nucleotide sequence may comprise a codon encoding arginine at a position corresponding to position 347 of SEQ ID NO: 1, and a codon encoding alanine, leucine, isoleucine or valine at a position corresponding to position 384 of SEQ ID NO: 1.

The Factor IX nucleotide sequence may comprise a codon encoding a large positively-polarised amino acid at a position corresponding to position 347 of SEQ ID NO: 1, and a codon encoding alanine or leucine at a position corresponding to position 384 of SEQ ID NO: 1. The Factor IX nucleotide sequence may comprise a codon encoding arginine, histidine or glutamine at a position corresponding to position 347 of SEQ ID NO: 1, and a codon encoding alanine or leucine at a position corresponding to position 384 of SEQ ID NO: 1. The Factor IX nucleotide sequence may comprise a codon encoding arginine at a position corresponding to position 347 of SEQ ID NO: 1, and a codon encoding alanine or leucine at a position corresponding to position 384 of SEQ ID NO: 1.

### The Factor IX Nucleotide Sequence may be Operably Linked to a Transcription Regulatory Element

The Factor IX nucleotide sequence may be operably linked to a transcription regulatory element.

Any appropriate transcription regulatory element may be used, such as HLP2, HLP1, LP1, HCR-hAAT, ApoE-hAAT, and LSP, which are all liver specific transcription regulatory elements. These transcription regulatory elements are described in more detail in the following references: HLP1: McIntosh J. et al., Blood 2013 Apr. 25, 121(17):3335-44; LP1: Nathwani et al., Blood. 2006 Apr. 1, 107(7): 2653-2661; HCR-hAAT: Miao et al., Mol Ther. 2000;1: 522-532; ApoE-hAAT: Okuyama et al., Human Gene Therapy, 7, 637-645 (1996); and LSP: Wang et al., Proc Natl Acad Sci U S A. 1999 Mar. 30, 96(7): 3906-3910. The HLP2 transcription regulatory element has a sequence of SEQ ID NO: 9.

The transcription regulatory element may comprise a promoter and/or an enhancer, such as the promoter element and/or enhancer element from HLP2, HLP1, LP1, HCR-hAAT, ApoE-hAAT, and LSP. Each of these transcription regulatory elements comprises a promoter, an enhancer, and optionally other nucleotides.

In an embodiment, the transcription regulatory element comprises an enhancer which is the human apolipoprotein E (ApoE) hepatic locus control region (HCR; Miao et al (2000), Molecular Therapy 1(6):522), or a fragment thereof. In an embodiment, the transcription regulatory element comprises a fragment of the HCR enhancer which is a fragment of at least 80, at least 90, at least 100, less than 192, between 80 and 192, between 90 and 192, between 100 and 250, or between 117 and 192 nucleotides in length. Optionally, the fragment of the HCR enhancer is between 100 and 250 nucleotides in length.

In an embodiment, the transcription regulatory element comprises a promoter which is a human alpha-1 anti-trypsin promoter (A1AT; Miao et al (2000), Molecular Therapy 1(6):522), or a fragment thereof. Optionally, the transcription regulatory element comprises a fragment of an A1AT promoter which is at least 100, at least 120, at least 150, at least 180, less than 255, between 100 and 255, between 150 and 225, between 150 and 300, or between 180 and 255 nucleotides in length. Optionally, the fragment of an A1AT promoter is between 150 and 300 nucleotides in length.

If the polynucleotide is intended for expression in the liver, the promoter may be a liver-specific promoter. Optionally, the promoter is a human liver-specific promoter. A “liver-specific promoter” is a promoter that drives a higher level of expression in liver cells compared to other cells in general. For example, the skilled person can determine whether a promoter is a liver-specific promoter by comparing expression of the polynucleotide in liver cells (such as Huh 7 cells) with expression of the polynucleotide in cells from other tissues. If the level of expression is higher in the liver cells, compared to the cells from other tissues, the promoter is a liver-specific promoter. Optionally, a liver-specific promoter does not drive an appreciable level of expression in non-liver cells.

### A Viral Particle Comprising the Polynucleotide

The invention further provides a viral particle comprising a recombinant genome comprising a polynucleotide of the invention. For the purposes of the present invention, the term “viral particle” refers to all or part of a virion. For example, the viral particle comprises a recombinant genome and may further comprise a capsid. The viral particle may be a gene therapy vector. Herein, the terms “viral particle” and “vector” are used interchangeably. For the purpose of the present application, a “gene therapy” vector is a viral particle that can be used in gene therapy, i.e. a viral particle that comprises all the required functional elements to express a transgene, such as a Factor IX nucleotide sequence, in a host cell after administration.

Suitable viral particles include a parvovirus, a retrovirus, a lentivirus, or a herpes simplex virus. The parvovirus may be an adeno-associated virus (AAV). The viral particle is preferably a recombinant adeno-associated viral (AAV) vector or a lentiviral vector. More preferably, the viral particle is an AAV viral particle. The terms AAV and rAAV are used interchangeably herein, unless the context indicates otherwise.

The genomic organization of all known AAV serotypes is very similar. The genome of AAV is a linear, single-stranded DNA molecule that is less than about 5,000 nucleotides in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

The recombinant viral genome of the invention may comprise ITRs. It is possible for an AAV vector of the invention to function with only one ITR. Thus, the viral genome comprises at least one ITR, but, more typically, two ITRs (generally with one either end of the viral genome, i.e. one at the 5′ end and one at the 3′ end). There may be intervening sequences between the polynucleotide and one or more of the ITRs. The polynucleotide of the invention may be incorporated into a viral particle located between two regular ITRs or located on either side of an ITR engineered with two D regions.

AAV sequences that may be used in the present invention for the production of AAV vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chiorini et al, 1997; Srivastava et al, 1983; Chiorini et al, 1999; Rutledge et al, 1998; and Wu et al, 2000. AAV serotype 1, 2, 3, 3B, 4, 5, 6, 7, 8, 9, 10, 11 or 12 may be used in the present invention. The sequences from the AAV serotypes may be mutated or engineered when being used in the production of gene therapy vectors.

Optionally, an AAV vector comprises ITR sequences which are derived from AAV1, AAV2, AAV4 and/or AAV6. Preferably the ITR sequences are AAV2 ITR sequences. Herein, the term AAVx/y refers to a viral particle that comprises some components from AAVx (wherein x is a AAV serotype number) and some components from AAVy (wherein y is the number of the same or different serotype). For example, an AAV2/8 vector may comprise a portion of a viral genome, including the ITRs, from an AAV2 strain, and a capsid derived from an AAV8 strain.

In an embodiment, the viral particle is an AAV viral particle comprising a capsid. AAV capsids are generally formed from three proteins, VP1, VP2 and VP3. The amino acid sequence of VP1 comprises the sequence of VP2. The portion of VP1 which does not form part of VP2 is referred to as VP1 unique or VP1U. The amino acid sequence of VP2 comprises the sequence of VP3. The portion of VP2 which does not form part of VP3 is referred to as VP2unique or VP2U. Preferably the capsid is an AAV5 capsid or a Mut C capsid, such as that disclosed in WO2016/181123.

A viral particle of the invention may be a “hybrid” particle in which the viral ITRs and viral capsid are from different parvoviruses, such as different AAV serotypes. Preferably, the viral ITRs and capsid are from different serotypes of AAV, in which case such viral particles are known as transcapsidated or pseudotyped. Likewise, the parvovirus may have a “chimeric” capsid (e. g., containing sequences from different parvoviruses, preferably different AAV serotypes) or a “targeted” capsid (e. g., a directed tropism).

In some embodiments, the recombinant AAV genome comprises intact ITRs, comprising functional terminal resolution sites (TRS). Such an AAV genome may contain one or two resolvable ITRs, i.e. ITRs containing a functional TRS at which site-specific nicking can take place to create a free 3′ hydroxyl group which can serve as a substrate for DNA polymerase to unwind and copy the ITR. Preferably, the recombinant genome is single-stranded (i.e., it is packaged into the viral particle in a single-stranded form). Optionally, the recombinant genome is not packaged in self-complementary configuration, i.e. the genome does not comprise a single covalently-linked polynucleotide strand with substantial self-complementary portions that anneal in the viral particle. Alternatively, the recombinant genome may be packaged in “monomeric duplex” form. “Monomeric duplexes” are described in WO 2011/122950. The genome may be packaged as two substantially complementary but non-covalently linked polynucleotides which anneal in the viral particle.

The viral particle may further comprise a poly A sequence. The poly A sequence may be positioned downstream of the nucleotide sequence encoding a functional Factor IX protein. The poly A sequence may be a bovine growth hormone poly A sequence (bGHpA). The poly A sequence may be between 250 and 270 nucleotides in length.

### Compositions, Methods and Uses

In a further aspect of the invention, there is provided a composition comprising the modified Factor IX polypeptide, polynucleotide, or vector/viral particle of the invention and a pharmaceutically acceptable excipient.

The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

The invention further provides a modified Factor IX polypeptide, a polynucleotide, a vector/viral particle or a composition of the invention for use in a method of treatment. Optionally, the method of treatment comprises administering an effective amount of the modified Factor IX polypeptide, polynucleotide, vector/viral particle, or composition of the invention to a patient.

The invention further provides a method of treatment comprising administering an effective amount of the modified Factor IX polypeptide, polynucleotide, composition, or vector/viral particle of the invention to a patient.

The invention further provides use of the modified Factor IX polypeptide, polynucleotide, vector/viral particle or composition of the invention in the manufacture of a medicament for use in a method of treatment. Optionally, the method of treatment comprises administering an effective amount of the modified Factor IX polypeptide, polynucleotide, composition or vector/viral particle of the invention to a patient. Optionally, the method of treatment is a gene therapy. A “gene therapy” involves administering a vector/viral particle of the invention that is capable of expressing a transgene (such as a polynucleotide of the invention) in the host to which it is administered.

Optionally, the method of treatment is a method of treating a coagulopathy such as haemophilia (for example haemophilia A or B) or Van Willebrands' disease. Preferably, the coagulopathy is characterised by increased bleeding and/or reduced clotting. Optionally, the method of treatment is a method of treating haemophilia, for example haemophilia B. In some embodiments, the patient is a patient suffering from haemophilia B. Optionally the patient has antibodies or inhibitors to Factor IX. Optionally, the modified Factor IX polypeptide, polynucleotide, composition and/or vector/viral particle is administered intravenously. Optionally, the modified Factor IX polypeptide, polynucleotide, composition and/or vector/viral particle is for administration only once (i.e. a single dose) to a patient.

When haemophilia B is “treated” in the above method, this means that one or more symptoms of haemophilia are ameliorated. It does not mean that the symptoms of haemophilia are completely remedied so that they are no longer present in the patient, although in some methods, this may be the case. The method of treatment may result in one or more of the symptoms of haemophilia B being less severe than before treatment. Optionally, relative to the situation pre-administration, the method of treatment results in an increase in the amount/concentration of circulating modified Factor IX polypeptide in the blood of the patient, and/or the overall level of modified Factor IX polypeptide activity detectable within a given volume of blood of the patient, and/or the specific activity (activity per amount of modified Factor IX polypeptide) of the modified Factor IX polypeptide in the blood of the patient.

A “therapeutically effective amount” refers to an amount effective, at dosages and for periods of time necessary, to achieve the desired therapeutic result, such as raising the level of functional modified factor IX polypeptide in a subject (so as to lead to functional modified factor IX polypeptide production at a level sufficient to ameliorate the symptoms of haemophilia B).

Optionally, the vector/viral particle is administered at a dose of less than 1×10¹¹, less than 1×10¹², less than 5×10¹², less than 2×10¹², less than 1.5×10¹², less than 3×10¹², less than 1×10¹³, less than 2×10¹³, or less than 3×10¹³ vector genomes per kg of weight of patient. Optionally, the dose of vector/viral particle that is administered is selected such that the subject expresses Factor IX at an activity of 10%-90%, 20%-80%, 30%-70%, 25%-50%, 20%-150%, 30%-140%, 40%-130%, 50%-120%, 60%-110% or 70%-100% of the Factor IX activity of a non-haemophilic healthy subject.

### SEQUENCE LISTING

TABLE 1

| Sequence | |
|---|---|
| identity number | Sequence description |
| 1 | Wild type Mälmo B immature FIX polypeptide |
| 2 | Wild type Mälmo B mature FIX polypeptide |
| 3 | Wild type Mälmo A immature FIX polypeptide |
| 4 | Wild type Mälmo A mature FIX polypeptide |
| 5 | Mälmo B immature FIX polypeptide with a |
| | K347R mutation |
| 6 | Mälmo B immature FIX polypeptide with an |
| | R384A mutation |
| 7 | Mälmo B immature FIX polypeptide with an |
| | R384L mutation |
| 8 | FIX-encoding polynucleotide sequence |
| 9 | Transcription regulatory sequence |

### Example 1

### In Vivo Expression and Assessment of FIX Variants

### Materials and Methods

### Generation of FIX Variants

The cDNA of codon optimized human FIX (hFIXco; (SEQ ID NO: 8), encoding wild type (WT) Malmo B variant FIX protein) was cloned into the pAV-sc-LP1 plasmid. Human FIX (hFIX) variants (K347L, K347R and R384L) were generated by site directed mutagenesis of the pAV-sc-LP1-hFIXco template using the Q5 Site-Directed Mutagenesis kit (NEB) following manufacturer's instructions and sequenced for consistency.

### Generation of AAV Vectors

The AAV self-complementary AAV cassette sc-LP1-hFIXco was pseudotyped with AAV8 and vector was generated by triple plasmid transfection of HEK 293T cells using

PEI. Cell pellet and supernatant were harvested 72 hours later and purified by affinity chromatography using AVB Sepharose High Performance (GE Healthcare) (Binny, C J, and AC Nathwani. 2012. “Vector Systems for Prenatal Gene Therapy: Principles of Adeno-Associated Virus Vector Design and Production.” Methods in Molecular Biology, 109-131. doi:10.1007/978-1-61779-873-3_6). AAV was dialysed into PBS overnight and stored at 4° C. and vector was titrated by alkaline agarose gel (Fagone, P, J F Wright, A C Nawthwani, A W Nienhuis, A M Davidoff, and J T Gray. 2012. “Systemic Errors in Quantitative Polymerase Chain Reaction Titration of Self-Complementary Adeno-Associated Viral Vectors and Improved Alternative Methods.” Human Gene Therapy Methods. doi: 10.1089/hgtb.2011.104).

### In Vivo Assessment

AAV vectors (1×10¹⁰vg/mouse) were injected into the tail vein of male 6 to 8-week-old FIX (haemophilia B) knock out mice (CL57B6) on the 129/sv background (Wang, L, M Zoppe, T M Hackeng, J H Griffin, K -F Lee, and IM Verma. 1997. “A factor IX-deficient Mouse Model of hemophilia B gene therapy.” PNAS 94: 11563-11566). Blood samples were collected from the tail vein into 4% sodium citrate (1:9 ratio) at 2-, 4-, 8- and 13-weeks post injection and the tail veins were cauterized after each collection. FIX antigen was measured by Asserachrom IX:Ag ELISA kit (Diagnostica Stago) following manufacturer's instructions. FIX activity was measured by chromogenic assay (Biophen Factor IX, Quadratech, UK), following manufacturer's instructions for end point quantification.

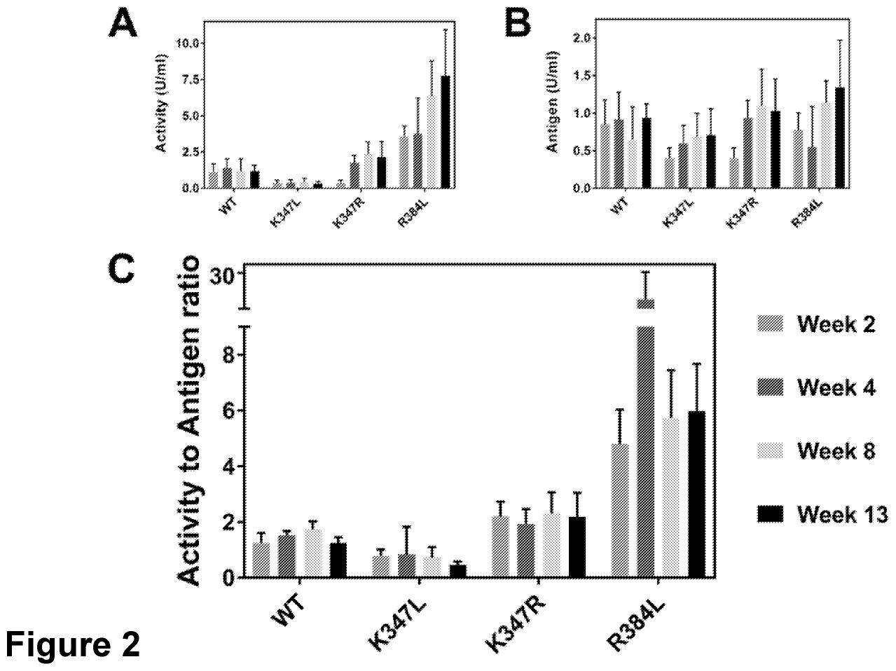

As shown in FIG. 2B, thirteen weeks post injection plasma FIX antigen levels were similar in mice transduced with vectors carrying either WT (0.93±0.19 U/ml), K347L (0.71±0.35 U/ml), K347R (1.03±0.43 U/ml) and K384L (1.34±0.63 U/ml). As shown in FIG. 2A, at thirteen weeks chromogenic FIX activity levels in plasma were higher in the mice injected with vector carrying K347R (2.14±1.07 U/ml) relative to WT (1.19±0.38 U/ml). In addition, activity was decreased in plasma from mice injected with K347L (0.31±0.16 U/ml) but increased with K384L (7.75±3.19 U/ml) relative to WT. The specific activity was obtained by dividing the activity level over the antigen level. As shown in FIG. 2C, specific activity in plasma from K347R mice (2.18±0.87) was more than 1.7-fold higher compared to plasma from mice in the WT group (1.25±0.20). On the other hand, the K347L (0.46±0.12) mutation led to a 2.7-fold reduced FIX activity relative to WT. Consistent with previous reports, specific activity of R384L (5.97±1.70) was 4.6-fold higher compared to WT. These results show that mutation of the FIX polypeptide chain at position K347 potentially increases overall specific activity of FIX.

Table 2 describes relative specific activities of r-hFIX K347R, r-hFIX K347L and r-hFIX-R384L.

TABLE 2

| | WT |
|---|---|
| K347R | 1.7 fold increased |
| K347L | 2.7 fold reduced |
| R384L | 4.6 fold increased |

### Example 2

### In Vitro Stable Expression and Assessment of FIX Variants

### Materials and Methods

### Generation of FIX Variants

The cDNA of codon optimized human FIX (‘hFIXco’ (SEQ ID NO: 8), encoding wild type (WT) Malmo B variant FIX protein) was cloned into the pcDNA5/FRT/TO vector (Thermo Scientific, Waltham, MA USA). Human FIX variants (K347R, R384A, R384L, K347R+R384L and K347R+R384A) were generated by site-directed mutagenesis of the pcDNA5/FRT-hFIXco template using the Q5 Site-Directed Mutagenesis kit (NEB) following manufacturer's instructions and sequenced for consistency.

### Generation of Stable Cell Lines

Site specific genome integration of FIX variants was carried out by co-transfection of Flp-In™ HEK293 cells (Thermo Scientific) with the pcDNA5/FRT/TO vector (containing gene of interest) and pOG44 vector (Thermo Scientific) containing the Flp recombinase. Cell lines stably expressing recombinant hFIX (r-hFIX) variants were obtained by selecting for the pcDNA5/FRT/TO antibiotic selection marker using culture media containing 50 μg/ml Hygromycin.

### In Vitro Assessment

Cells (1.5×10⁶) stably expressing r-hFIX variants were seeded in triplicate in T25 cell culture flasks and cultured for 24 hours before switching to serum free expression media containing 10 μg/ml Vitamin K. Expression media was conditioned for 48 hours on cells, harvested, subsampled and frozen at −80° C. pending analysis. FIX antigen was measured by Asserachrom IX:Ag ELISA kit (Diagnostica Stago) following manufacturer's instructions. FIX activity was measured by chromogenic assay (Biophen Factor IX, Quadratech, UK), following manufacturer's instructions for end point quantification.

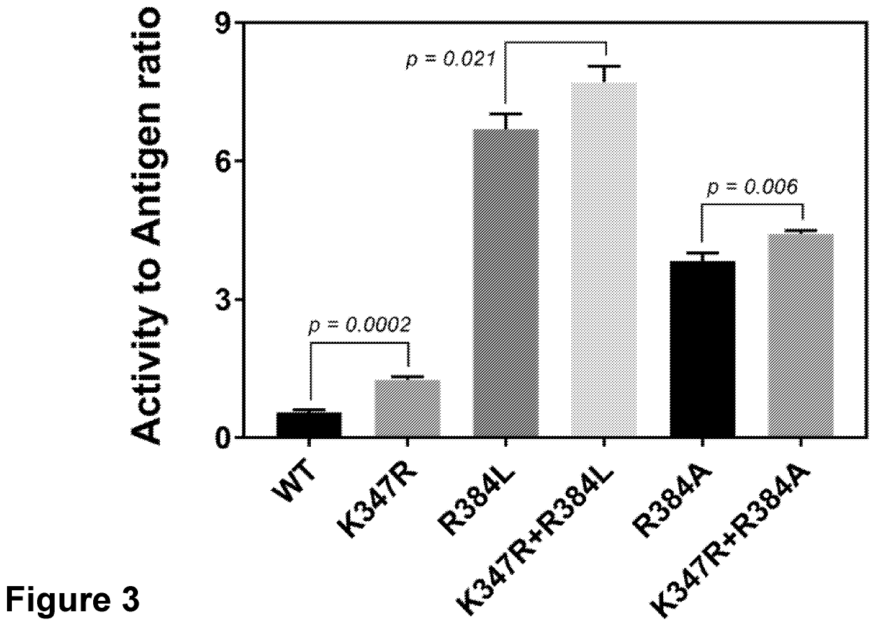

As shown in FIG. 3 and Table 3, specific activity was 2.3-fold higher in variant r-hFIX-K347R (1.25±0.08) relative to r-hFIX-WT (0.54±0.06). In addition, specific activity in r-hFIX-R384L (6.69±0.32) and r-hFIX R384A (3.38±0.17) were 12.4-fold and 6.3-fold increased relative to WT. Also, specific activity of double mutants r-hFIX-K347R+R384L (7.71±0.35) and r-hFIX-K347R+R384A (4.42±0.17) correspondingly increased 14.3-fold and 8.2-fold relative to r-hFIX-WT. Moreover, specific activity of the double mutant r-hFIX-K347R+R384L was 1.2-fold higher relative to R384L, and specific activity of the double mutant r-hFIX-K347R+R384A was 1.3-fold higher in the relative to r-hFIX-R384A. These results show that mutations at position K347 can be combined with mutations at position R384 of the FIX polypeptide chain to increase overall specific activity of FIX.

Table 3 describes relative specific activities for r-hFIX-K347R, r-hFIX-K347R+R384A and r-hFIX-K347+R384L.

TABLE 3

| | WT | K347R | R384A | R384L |
|---|---|---|---|---|
| K347R | 2.3 fold | — | — | — |
| K347R + R384A | 8.2 fold | 3.6 fold | 1.3 fold | — |
| K347R + R384L | 14.3 fold | 6.2 fold | 2.3 fold | 1.2 fold |

### Example 3

### Characterization of Purified FIX Variants

### Materials and Methods

### Expression of Recombinant FIX Variants

Flp-In HEK293 cell lines stably expressing wild-type recombinant human FIX (r-FIX-WT), or recombinant human FIX variants (K347R, R384L, K347R/R384L) were expanded into a 6320 cm² cell factory (Thermo Scientific, Waltham, Mass., USA) and conditioned for 24 h in FIX-specific expression media (Dulbecco's modified Eagle's medium/F-12 without phenol red supplemented with 2 mM L-glutamine, 100 U/ml penicillin, 0.1 mg/ml streptomycin, 0.25 μg/ml amphotericin B, 50 μg/ml Hygromycin, 10 μg/ml ITS, and 6μg/ml vitamin K). Conditioned media was collected for 8 consecutive days, filtered over an 0.45 μm polyethersulfone membrane, and supplemented with 1 mM benzamidine prior to storage at −20° C.

### Purification of Recombinant Human FIX

Conditioned media (16 litre) was thawed at 37° C., applied to a size 6 A ultrafiltration hollow fiber cartridge using an Akta flux 6 instrument (GE Healthcare), diafiltrated to ˜500 ml in 20 mM Hepes, 0.15 M NaCl, pH 7.4 (HBS), and stored at −20° C. Following thawing at 37° C., the pool was applied at ambient temperatures to a 4.8×4 cm Q Sepharose Fast Flow column (GE Healthcare) equilibrated in 20 mM Tris, 0.15 M NaCl, pH 7.4. Following washing with the same buffer, bound protein was eluted with a linear 0.15-0.75 M NaCl gradient. Fractions containing FIX activity were stored at −80° C. Following thawing at 37° C., fractions containing FIX activity were pooled and dialyzed at 4° C. overnight to 20 mM Tris, 0.15 M NaCl, pH 7.4. The dialysate was applied at ambient temperatures to a 5 ml IXSelect column (GE Healthcare) equilibrated in the same buffer. Following washing with 20 mM Tris, 0.50 M NaCl, pH 7.4, bound protein was eluted with by isocratic elution using 20 mM Tris, 2 M MgCl2, pH 7.4 at a flow rate of 4.00 ml/min. Fractions containing FIX activity were immediately pooled and dialyzed once at 4° C. for 2 hours to 20 mM Tris (4 litre) and once for 2 hours to 50 mM sodium phosphate, pH 6.8 (4 litre) and again overnight to 50 mM sodium phosphate, pH 6.8 (4 litre). The dialysate was applied at ambient temperatures to a Bio-Scale CHTS-I hydroxyapatite column (Bio-Rad, Hercules, Calif., USA) equilibrated in 50 mM sodium phosphate, pH 6.8. Following washing with the same buffer, bound protein was first eluted with a linear 50-300 mM Sodium phosphate gradient at a flow rate of 1.25 ml/min. Fractions containing FIX activity were analyzed employing SDS-PAGE analysis, stored at −80° C., pooled upon thawing at 37° C., ultrafiltrated employing Amicon Ultra-15 centrifugal filter units (Merck, N.J., USA) with 30 kDa molecular weight cutoff to 5-10 mg ml⁻¹ in HBS, 50% (v/v) glycerol, and stored at −20° C. The concentration of purified recombinant hFIX was determined spectrophotometrically from absorbance at 280 nm, after correction for the background at 320 nm (absorption=A280-1.7xA320) using an extinction coefficient (E_{0.1%}, 280 nm) of 1.32. The typical yield of fully y-carboxylated recombinant FIX was 0.3 mg/litre conditioned medium. Purity of the purified product was visualized by Coomassie Brilliant Blue staining employing SDS-PAGE analysis and was confirmed to be equal or higher than 99%.

### One-Stage Clotting and Chromogenic FIX Activity Assay

r-hFIX variants were spiked in duplicate into FIX-depleted plasma (HemosIL, Instrumentation Laboratory (IL), MA, USA) at 0.25 μg/ml, aliquoted and stored at −80° C. Upon thawing, one-stage clotting activity (APTT) was measured in triplicate using SynthaSIL APTT reagent (HemosIL, IL) on the automated ACL TOP 700 coagulometer (IL). In addition, chromogenic activity of spiked plasma samples was measured in triplicate on the automated ACL TOP 700 coagulometer (IL) using a commercial FIX chromogenic assay kit (Rox Factor IX, Rossix, Sweden). Both assays were calibrated using a frozen plasma standard (CRYOcheck Normal Reference plasma, Precision Biologic, Canada).

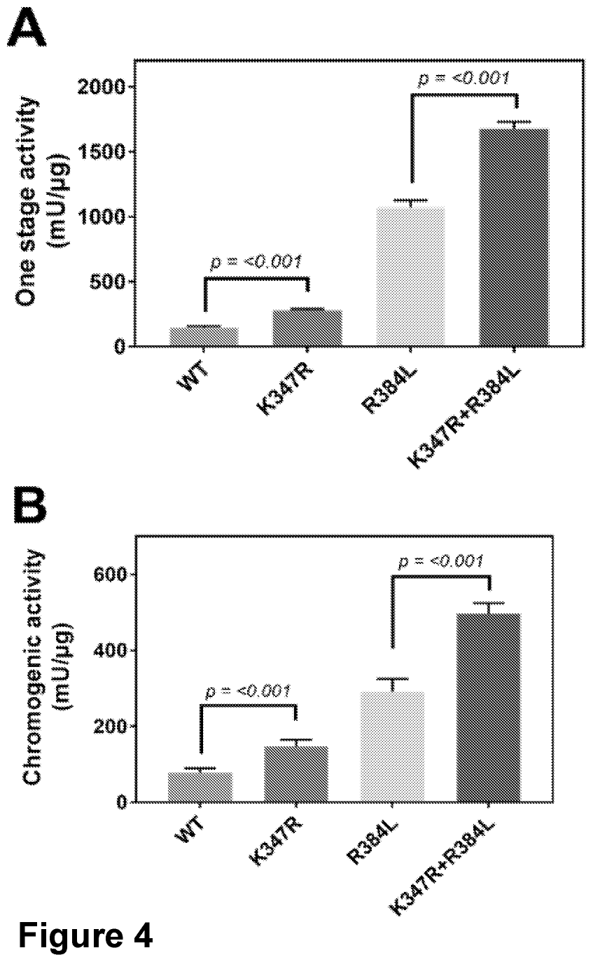

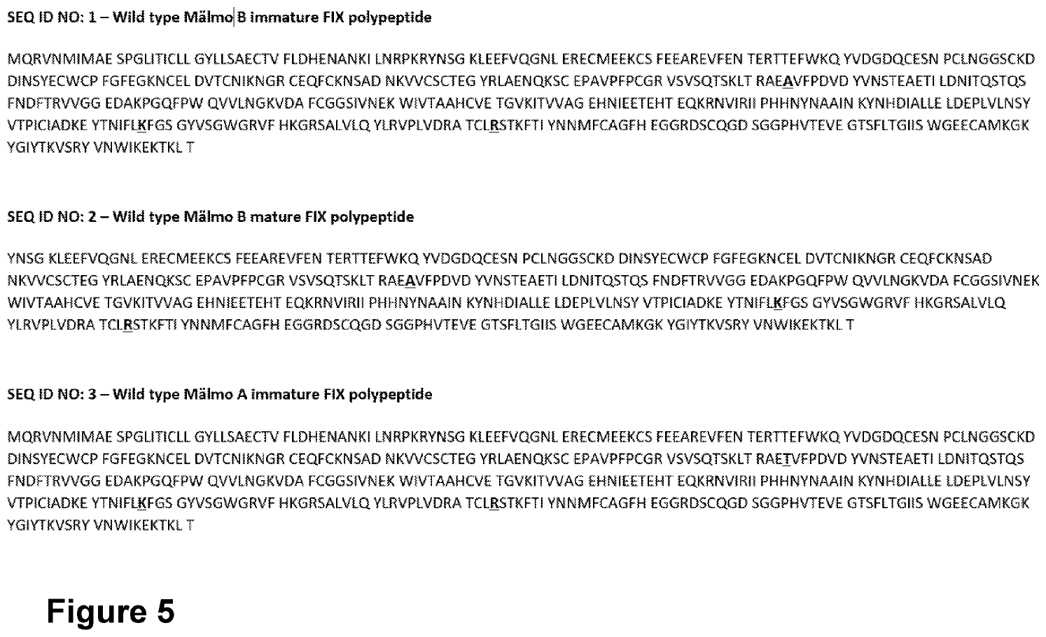

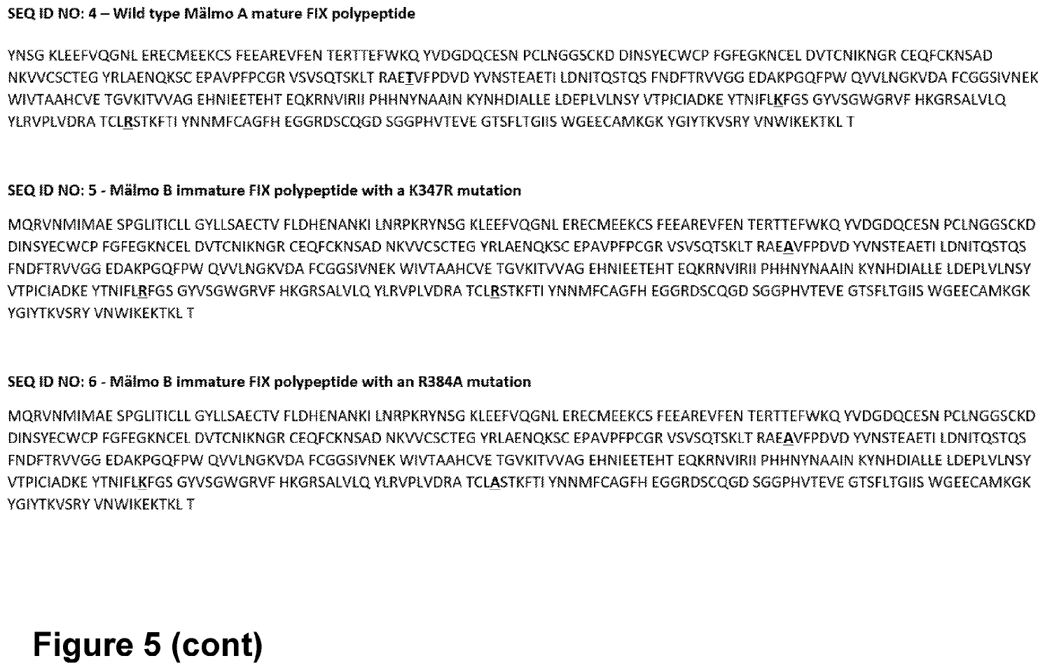

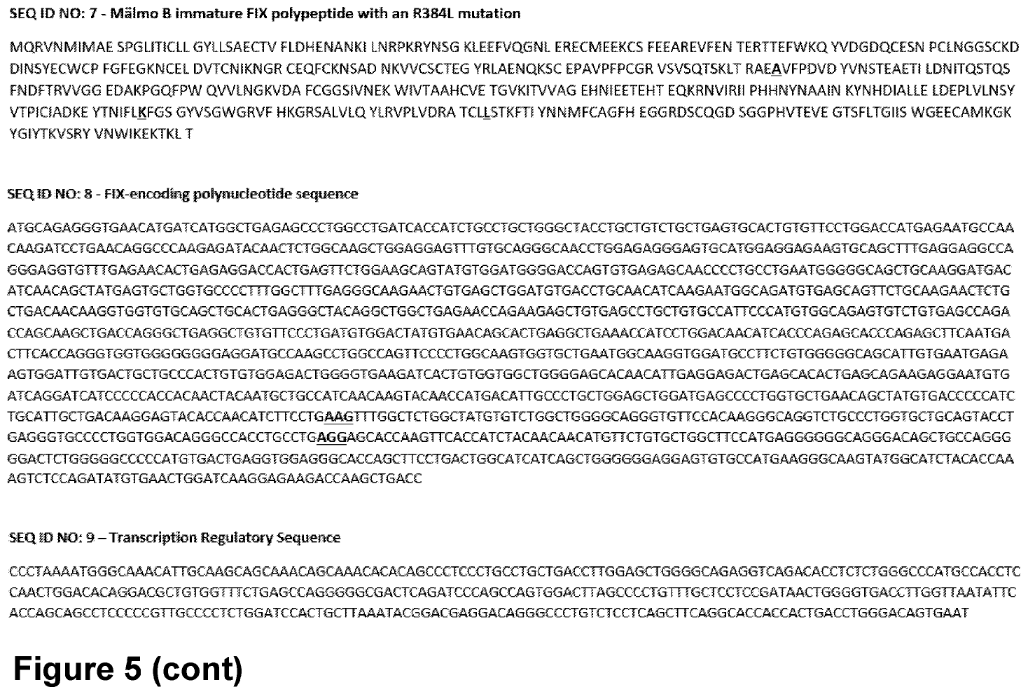

As shown in FIG. 4A, assaying of one-stage clotting activity revealed that specific activity was 1.9-fold higher for the variant r-hFIX-K347R (279±22 mU/μg) relative to r-hFIX-WT (144±13 mU/μg). In addition, specific clotting of r-hFIX-R384L (1071±56 mU/μg) was 7.4-fold higher relative to r-hFIX-WT. Finally, specific activity of the double mutant r-hFIX-K347R+R384L (1677±52 mU/μg) was 11.6-fold higher relative to r-hFIX-WT and 1.6-fold higher relative to r-hFIX-R384L. These results show that the r-hFIX-K347R mutation increases activity of both the r-hFIX-WT as well as the r-hFIX-R384L mutant. The one-stage clotting assay has historically been used as the gold-standard for clinical readout of FIX activity. The chromogenic FIX assay has recently been adopted in research as alternative to the one-stage clotting assay. As shown in FIG. 4B, assaying of chromogenic FIX activity revealed that specific activity was 1.9-fold higher for r-hFIX-K347R (147±18 mU/μg) relative to r-hFIX-WT (78±12 mU/μg), confirming previously observed trends in fold change of r-hFIX-K347 over r-hFIX-WT. However, specific chromogenic activity of both r-hFIX-WT and r-hFIX-K347R was lower in the chromogenic assay as compared to the values obtained using the one-stage clotting assay. Discrepancies in FIX activity between the one-stage clotting assay and chromogenic assay were also apparent when assaying gain-of-function FIX variants r-hFIX-R384L and r-hFIX-K347R+R384L. For example, specific activity of r-hFIX-R384L (292±33 mU/μg) was 3.7-fold higher relative to r-hFIX-WT, while specific activity of r-hFIX-K347R+R384L (497±28 mU/μg) was 6.4-fold higher relative to r-hFIX-WT. In addition, specific activity of r-hFIX-K347R+R384L was 1.7-fold increased relative to r-hFIX-R384L. These results confirm higher activity for r-hFIX-K347R, r-hFIX-R384L and r-hFIX-K347R+R384L mutations over the wildtype FIX protein when tested by means of chromogenic FIX activity.

Table 4 describes relative specific activities of r-hFIX K347R, r-hFIX K347L and r-hFIX K347R+R384L as determined using a one-stage clotting assay.

TABLE 4

| | WT | K347R | R384L |
|---|---|---|---|
| K347R | 1.9 fold | — | — |
| R384L | 7.4 fold | — | — |
| K347R + R384L | 11.6 fold | 6.0 fold | 1.6 fold |

Table 5 describes relative specific activities of r-hFIX K347R, r-hFIX R384L and r-hFIX K347R+R384L as determined using a chromogenic assay.

TABLE 5

| | WT | K347R | R384L |
|---|---|---|---|
| K347R | 1.9 fold | — | — |
| R384L | 3.7 fold | — | — |
| K347R + R384L | 6.4 fold | 3.4 fold | 1.7 fold |

The invention described herein also relates to the following aspects:

1. A modified Factor IX polypeptide comprising a mutation at a position corresponding to position 347 of SEQ ID NO: 1.

2. The modified Factor IX polypeptide of aspect 1, wherein the mutation at a position corresponding to position 347 of SEQ ID NO: 1 is a gain-of-function mutation.

3. The modified Factor IX polypeptide of aspect 1 or 2, wherein the modified Factor IX polypeptide has a higher activity compared to an equivalent polypeptide lacking a mutation at a position corresponding to position 347 of SEQ ID NO: 1.

4. The modified Factor IX polypeptide of any one of aspects 1-3, wherein the modified Factor IX polypeptide has a higher activity compared to a polypeptide of SEQ ID NO: 1, SEQ ID NO: 2, SEQ ID NO: 3, or SEQ ID NO: 4.

5. The modified Factor IX polypeptide of any one of the preceding aspects, wherein the modified Factor IX polypeptide has a higher specific activity compared to an equivalent polypeptide lacking a mutation at a position corresponding to position 347 of SEQ ID NO: 1.

6. The modified Factor IX polypeptide of any one of the preceding aspects, wherein the modified Factor IX polypeptide has a higher specific activity compared to a polypeptide of SEQ ID NO: 1, SEQ ID NO: 2, SEQ ID NO: 3, or SEQ ID NO: 4.

7. The modified Factor IX polypeptide of aspect 5 or 6, wherein the specific activity is measured using a chromogenic assay.

8. The modified Factor IX polypeptide of aspect 7, wherein the modified Factor IX polypeptide has an at least 1.2 fold, at least 1.5 fold, at least 1.6 fold, at least 1.7 fold, or at least 1.8 fold higher specific activity compared to an equivalent polypeptide lacking a mutation at a position corresponding to position 347 of SEQ ID NO: 1, or compared to a polypeptide of SEQ ID NO: 1, SEQ ID NO: 2, SEQ ID NO: 3, or SEQ ID NO: 4.

9. The modified Factor IX polypeptide of one of aspects 5-8, wherein the specific activity is measured using a clotting assay.

10. The modified Factor IX polypeptide of aspect 9, wherein the modified Factor IX polypeptide has an at least 1.2 fold, at least 1.5 fold, at least 1.6 fold, at least 1.7 fold, or at least 1.8 fold higher specific activity compared to an equivalent polypeptide lacking a mutation at a position corresponding to position 347 of SEQ ID NO: 1, or compared to a polypeptide of SEQ ID NO: 1, SEQ ID NO: 2, SEQ ID NO: 3, or SEQ ID NO: 4.

11. The modified Factor IX polypeptide of any one of the preceding aspects, wherein the mutation at a position corresponding to position 347 of SEQ ID NO: 1 is a mutation to a large positively-polarised amino acid.

12. The modified Factor IX polypeptide of any one of the preceding aspects, wherein the mutation at a position corresponding to position 347 of SEQ ID NO: 1 is a mutation to an amino acid residue selected from the group consisting of arginine, histidine, and glutamine.

13. The modified Factor IX polypeptide of aspect 12, wherein the mutation at a position corresponding to position 347 of SEQ ID NO: 1 is a mutation to arginine.

14. The modified Factor IX polypeptide of any one of the preceding aspects, wherein the modified Factor IX polypeptide further comprises a mutation at a position corresponding to position 384 of SEQ ID NO: 1.

15. The modified Factor IX polypeptide of aspect 14, wherein the mutation at a position corresponding to position 384 of SEQ ID NO: 1 is a mutation to a small non-polar amino acid.

16. The modified Factor IX polypeptide of aspect 14 or 15, wherein the mutation at a position corresponding to position 384 of SEQ ID NO: 1 is a mutation to an amino acid selected from the group consisting of alanine, valine, leucine and isoleucine.

17. The modified Factor IX polypeptide of aspect 16, wherein the mutation at a position corresponding to position 384 of SEQ ID NO: 1 is a mutation to alanine.

18. The modified Factor IX polypeptide of aspect 16, wherein the mutation at a position corresponding to position 384 of SEQ ID NO: 1 is a mutation to leucine.

19. The modified Factor IX polypeptide of aspect 14, wherein the mutation at a position corresponding to position 384 of SEQ ID NO: 1 is a mutation to glutamine or aspartate.

20. The modified Factor IX polypeptide of aspect 17, comprising a mutation to arginine at a position corresponding to position 347 and a mutation to alanine at a position corresponding to position 384 of SEQ ID NO: 1.

21. The modified Factor IX polypeptide of aspect 18, comprising a mutation to arginine at a position corresponding to position 347 and a mutation to leucine at a position corresponding to position 384 of SEQ ID NO: 1.

22. The modified Factor IX polypeptide of any one of aspects 14-21, wherein the modified Factor IX polypeptide has a higher specific activity compared to an equivalent polypeptide lacking a mutation at a position corresponding to position 347 of SEQ ID NO: 1 and lacking a mutation at a position corresponding to position 384 of SEQ ID NO: 1.

23. The modified Factor IX polypeptide of any one of the preceding aspects, wherein the modified Factor IX polypeptide has a higher specific activity compared to a polypeptide of SEQ ID NO: 5.

24. The modified Factor IX polypeptide of aspect 23, wherein the modified Factor IX polypeptide has an at least 2.5 fold, at least 3 fold, at least 3.3 fold, at least 4 fold, at least 4.5 fold, at least 5 fold, at least 5.5 fold, or at least 6 fold higher specific activity compared to a polypeptide of SEQ ID NO: 5.

25. The modified Factor IX polypeptide of any one of the preceding aspects, wherein the modified Factor IX polypeptide has a higher specific activity compared to a polypeptide of SEQ ID NO: 6.

26. The modified Factor IX polypeptide of aspect 25, wherein the modified Factor IX polypeptide has an at least 1.1 fold, at least 1.2 fold, at least 2.0 fold or at least 2.2 fold higher specific activity compared to a polypeptide of SEQ ID NO: 6.

27. The modified Factor IX polypeptide of any one of the preceding aspects, wherein the modified Factor IX polypeptide has a higher specific activity compared to a polypeptide of SEQ ID NO: 7.

28. The modified Factor IX polypeptide of aspect 27, wherein the modified Factor IX polypeptide has an at least 1.1 fold, at least 1.15 fold, at least 1.3 fold, at least 1.5 fold, or at least 1.6 fold higher specific activity compared to a polypeptide of SEQ ID NO: 7.

29. The modified Factor IX polypeptide of any one of aspects 22-28, wherein the specific activity is measured using a chromogenic assay or a clotting assay.

30. The modified Factor IX polypeptide of any one of the preceding aspects, wherein the modified Factor IX polypeptide comprises a polypeptide sequence at least 80%, at least 90%, at least 95%, at least 98%, or at least 99% identical to a fragment of SEQ ID NO: 1 or SEQ ID NO: 2 of at least 200, at least 250, at least 300, between 200 and 415, between 250 and 415, or between 300 and 415 amino acids.

31. The modified Factor IX polypeptide of any one of the preceding aspects, wherein the modified Factor IX polypeptide comprises a polypeptide sequence at least 80%, at least 90%, at least 95%, at least 98%, or at least 99% identical to SEQ ID NO: 1 or SEQ ID NO: 2.

32. The modified Factor IX polypeptide of aspect 30 or 31, wherein the modified Factor IX polypeptide comprises a polypeptide sequence at least 98% identical to a fragment of between 300 and 415 amino acids of SEQ ID NO: 1.

33. The modified Factor IX polypeptide of any one of aspects 30-32, wherein the modified Factor IX polypeptide comprises a polypeptide sequence at least 98% identical to a fragment of between 300 and 415 amino acids of SEQ ID NO: 2.

34. The modified Factor IX polypeptide of any one of aspects 30-33, wherein the modified Factor IX polypeptide comprises a polypeptide sequence at least 98% identical to SEQ ID NO: 1.

35. The modified Factor IX polypeptide of any one of aspects 30-34, wherein the modified Factor IX polypeptide comprises a polypeptide sequence at least 98% identical to SEQ ID NO: 2.

36. The modified FIX polypeptide of any one of aspects 1-13 or 30-35, wherein the modified Factor IX polypeptide comprises a polypeptide sequence that is identical to SEQ ID NO:1 or SEQ ID NO: 2, except that the modified Factor IX polypeptide comprises a mutation at a position corresponding to position 347 of SEQ ID NO: 1.

37. The modified FIX polypeptide of any one of the preceding aspects, wherein the modified Factor IX polypeptide comprises a polypeptide sequence that is identical to SEQ ID NO: 1 or SEQ ID NO: 2, except that the modified Factor IX polypeptide comprises a mutation at a position corresponding to position 347 of SEQ ID NO: 1 and a mutation at a position corresponding to position 384 of SEQ ID NO: 1.

38. A polynucleotide comprising a Factor IX nucleotide sequence that encodes a modified Factor IX polypeptide of any one of the preceding aspects.

39. The polynucleotide of aspect 38, wherein the Factor IX nucleotide sequence is at least 80%, at least 90%, at least 95%, at least 98%, or at least 99% identical to a fragment of at least 900, at least 1000, at least 1300, or at least 13800 nucleotides of SEQ ID NO: 8.

40. The polynucleotide of aspect 39, wherein the Factor IX nucleotide sequence is at least 80%, at least 90%, at least 95%, at least 98%, or at least 99% identical to SEQ ID NO: 8.

41. The polynucleotide of any one of aspects 38-40, wherein the Factor IX nucleotide sequence comprises a codon that encodes an amino acid at a position corresponding to position 347 of SEQ ID NO: 1, and wherein the codon that encodes an amino acid at a position corresponding to position 347 of SEQ ID NO: 1 encodes arginine.

42. The polynucleotide of aspect 41, wherein the codon that encodes an amino acid at a position corresponding to position 347 of SEQ ID NO: 1 is CGT, CGC, CGA, CGG, AGA or AGG.

43. The polynucleotide of aspect 42, wherein the codon that encodes an amino acid at a position corresponding to position 347 of SEQ ID NO: 1 is AGG.

44. The polynucleotide of any one of aspects 38-43, wherein the Factor IX nucleotide sequence comprises a codon that encodes an amino acid at a position corresponding to position 384 of SEQ ID NO: 1, and wherein the codon that encodes an amino acid at a position corresponding to position 384 encodes alanine.

45. The polynucleotide of aspect 44, wherein the codon that encodes an amino acid at a position corresponding to position 384 of SEQ ID NO: 1 is GCT, GCC, GCA or GCG.

46. The polynucleotide of aspect 44 or 45, wherein the codon that encodes an amino acid at a position corresponding to position 384 of SEQ ID NO: 1 is GCC.

47. The polynucleotide of any one of aspects 38-43, wherein the Factor IX nucleotide sequence comprises a codon that encodes an amino acid at a position corresponding to position 384 of SEQ ID NO: 1, and wherein the codon that encodes an amino acid at a position corresponding to position 384 encodes leucine.

48. The polynucleotide of aspect 47, wherein the codon that encodes an amino acid at a position corresponding to position 384 of SEQ ID NO: 1 is CTX, wherein X is any nucleotide.

49. The polynucleotide of aspect 47 or 48, wherein the codon that encodes an amino acid at a position corresponding to position 384 of SEQ ID NO: 1 is CTC or CTG.

50. The polynucleotide of any one of aspects 38-49, wherein the Factor IX nucleotide sequence is operably linked to a transcription regulatory element.

51. The polynucleotide of aspect 50, wherein the transcription regulatory element comprises a liver-specific promoter and/or an enhancer.

52. The polynucleotide of aspect 50 or 51, wherein the transcription regulatory element is at least 80%, at least 90%, at least 95%, at least 98%, or at least 99% identical to SEQ ID NO: 9.

53. A viral particle comprising a recombinant genome comprising the polynucleotide of any one of aspects 38-52.

54. The viral particle of aspect 53, which is an AAV, adenoviral, or lentiviral viral particle.

55. The viral particle of aspect 54, wherein the viral particle is an AAV viral particle.

56. The viral particle of any one of aspects 53-55, wherein the viral particle further comprises:
a) AAV2 ITRs;
b) a polyA sequence;
c) an origin of replication; and/or
d) two resolvable ITRs.

57. The viral particle of any one of aspects 53-56, wherein administration of the viral particle is associated with a shortened bleeding time in a mouse tail clip assay compared to administration of an equivalent dose of an equivalent viral particle comprising a Factor IX nucleotide sequence encoding a Factor IX polypeptide of SEQ ID NO: 1 or SEQ ID NO: 2.

58. A composition comprising the modified Factor IX polypeptide, polynucleotide or viral particle of any one of the preceding aspects and a pharmaceutically acceptable excipient.

59. The modified Factor IX polypeptide, polynucleotide, viral particle or composition of any one of the preceding aspects for use in a method of treatment.

60. The modified Factor IX polypeptide, polynucleotide, viral particle or composition for use of aspect 59, wherein the method of treatment comprises administering an effective amount of the modified Factor IX polypeptide, polynucleotide, viral particle or composition of any one of aspects 1-58 to a patient.

61. A method of treatment comprising administering an effective amount of the modified Factor IX polypeptide, polynucleotide, viral particle or composition of any one of aspects 1-58 to a patient.

62. Use of the modified Factor IX polypeptide, polynucleotide, viral particle or composition of any one of aspects 1-58 in the manufacture of a medicament for use in a method of treatment.

63. The use of aspect 62, wherein the method of treatment comprises administering an effective amount of the modified Factor IX polypeptide, polynucleotide, viral particle or composition of any one of aspects 1-58 to a patient.

64. The modified Factor IX polypeptide for use, polynucleotide for use, viral particle for use, composition for use or method of any one of aspects 59-63, wherein the method of treatment is a method of treating haemophilia.

65. The modified Factor IX polypeptide for use, polynucleotide for use, viral particle for use, composition for use or method of aspect 64, wherein the haemophilia is haemophilia B.

66. The modified Factor IX polypeptide for use, polynucleotide for use, viral particle for use, composition for use or method of aspect 65, wherein the patient has antibodies or inhibitors to Factor IX.

1. A modified Factor IX polypeptide comprising a mutation at a position corresponding to position 347 of SEQ ID NO: 1.

2. The modified Factor IX polypeptide of claim 1, wherein the mutation at a position corresponding to position 347 of SEQ ID NO: 1 is a gain-of-function mutation.

3. The modified Factor IX polypeptide of claim 1 or 2, wherein the modified Factor IX polypeptide has a higher activity compared:
(i) to an equivalent polypeptide lacking a mutation at a position corresponding to position 347 of SEQ ID NO: 1; and/or
(ii) to a polypeptide of SEQ ID NO: 1, SEQ ID NO: 2, SEQ ID NO: 3, or SEQ ID NO: 4.

4. The modified Factor IX polypeptide of any one of the preceding claims, wherein the modified Factor IX polypeptide has a higher specific activity compared to an equivalent polypeptide lacking a mutation at a position corresponding to position 347 of SEQ ID NO: 1.

5. The modified Factor IX polypeptide of any one of the preceding claims, wherein the modified Factor IX polypeptide has a higher specific activity compared to a polypeptide of SEQ ID NO: 1, SEQ ID NO: 2, SEQ ID NO: 3, or SEQ ID NO: 4.

6. The modified Factor IX polypeptide of claim 4 or 5, wherein the specific activity is measured using a chromogenic assay.

7. The modified Factor IX polypeptide of claim 6, wherein the modified Factor IX polypeptide has an at least 1.2 fold, at least 1.5 fold, at least 1.6 fold, at least 1.7 fold, or at least 1.8 fold higher specific activity compared to an equivalent polypeptide lacking a mutation at a position corresponding to position 347 of SEQ ID NO: 1, or compared to a polypeptide of SEQ ID NO: 1, SEQ ID NO: 2, SEQ ID NO: 3, or SEQ ID NO: 4.

8. The modified Factor IX polypeptide of one of claims 4-7, wherein the specific activity is measured using a clotting assay.

9. The modified Factor IX polypeptide of claim 8, wherein the modified Factor IX polypeptide has an at least 1.2 fold, at least 1.5 fold, at least 1.6 fold, at least 1.7 fold, or at least 1.8 fold higher specific activity compared to an equivalent polypeptide lacking a mutation at a position corresponding to position 347 of SEQ ID NO: 1, or compared to a polypeptide of SEQ ID NO: 1, SEQ ID NO: 2, SEQ ID NO: 3, or SEQ ID NO: 4.

10. The modified Factor IX polypeptide of any one of the preceding claims, wherein the mutation at a position corresponding to position 347 of SEQ ID NO: 1 is:
(i) a mutation to a large positively-polarised amino acid; or
(ii) a mutation to an amino acid residue selected from the group consisting of arginine, histidine, and glutamine.

11. The modified Factor IX polypeptide of any one of the preceding claims, wherein the mutation at a position corresponding to position 347 of SEQ ID NO: 1 is a mutation to arginine.

12. The modified Factor IX polypeptide of any one of the preceding claims, wherein the modified Factor IX polypeptide further comprises a mutation at a position corresponding to position 384 of SEQ ID NO: 1.

13. The modified Factor IX polypeptide of claim 12, wherein the mutation at a position corresponding to position 384 of SEQ ID NO: 1 is:
(i) a mutation to a small non-polar amino acid; or
(ii) a mutation to an amino acid selected from the group consisting of glutamine, aspartate, alanine, valine, leucine and isoleucine.

14. The modified Factor IX polypeptide of claim 13:
(i) wherein the mutation at a position corresponding to position 384 of SEQ ID NO: 1 is a mutation to alanine; or
(ii) wherein the mutation at a position corresponding to position 384 of SEQ ID NO: 1 is a mutation to leucine; or
(iii) comprising a mutation to arginine at a position corresponding to position 347 and a mutation to alanine at a position corresponding to position 384 of SEQ ID NO: 1; or
(iv) comprising a mutation to arginine at a position corresponding to position 347 and a mutation to leucine at a position corresponding to position 384 of SEQ ID NO: 1.

15. The modified Factor IX polypeptide of any one of claims 12-14, wherein the modified Factor IX polypeptide has a higher specific activity compared to an equivalent polypeptide lacking a mutation at a position corresponding to position 347 of SEQ ID NO: 1 and lacking a mutation at a position corresponding to position 384 of SEQ ID NO: 1.

16. The modified Factor IX polypeptide of any one of the preceding claims, wherein:
(i) the modified Factor IX polypeptide has a higher specific activity compared to a polypeptide of SEQ ID NO: 5, SEQ ID NO: 6 or SEQ ID NO: 7; and/or
(ii) the modified Factor IX polypeptide has an at least 2.5 fold, at least 3 fold, at least 3.3 fold, at least 4 fold, at least 4.5 fold, at least 5 fold, at least 5.5 fold, or at least 6 fold higher specific activity compared to a polypeptide of SEQ ID NO: 5; and/or
(iii) the modified Factor IX polypeptide has an at least 1.1 fold, at least 1.2 fold, at least 2.0 fold or at least 2.2 fold higher specific activity compared to a polypeptide of SEQ ID NO: 6; and/or
(iv) the modified Factor IX polypeptide has an at least 1.1 fold, at least 1.15 fold, at least 1.3 fold, at least 1.5 fold, or at least 1.6 fold higher specific activity compared to a polypeptide of SEQ ID NO: 7; and/or
(v) the specific activity is measured using a chromogenic assay or a clotting assay.

17. The modified Factor IX polypeptide of any one of the preceding claims, wherein:
(i) the modified Factor IX polypeptide comprises a polypeptide sequence at least 80%, at least 90%, at least 95%, at least 98%, or at least 99% identical to a fragment of SEQ ID NO: 1 or SEQ ID NO: 2 of at least 200, at least 250, at least 300, between 200 and 415, between 250 and 415, or between 300 and 415 amino acids; or
(ii) the modified Factor IX polypeptide comprises a polypeptide sequence at least 80%, at least 90%, at least 95%, at least 98%, or at least 99% identical to SEQ ID NO: 1 or SEQ ID NO: 2; or
(iii) the modified Factor IX polypeptide comprises a polypeptide sequence at least 98% identical to a fragment of between 300 and 415 amino acids of SEQ ID NO: 1; or
(iv) the modified Factor IX polypeptide comprises a polypeptide sequence at least 98% identical to a fragment of between 300 and 415 amino acids of SEQ ID NO: 2; or
(v) the modified Factor IX polypeptide comprises a polypeptide sequence at least 98% identical to SEQ ID NO: 1; or
(vi) the modified Factor IX polypeptide comprises a polypeptide sequence at least 98% identical to SEQ ID NO: 2; or
(vii) the modified Factor IX polypeptide comprises a polypeptide sequence that is identical to SEQ ID NO:1 or SEQ ID NO: 2, except that the modified Factor IX polypeptide comprises a mutation at a position corresponding to position 347 of SEQ ID NO: 1; or
(viii) the modified Factor IX polypeptide comprises a polypeptide sequence that is identical to SEQ ID NO: 1 or SEQ ID NO: 2, except that the modified Factor IX polypeptide comprises a mutation at a position corresponding to position 347 of SEQ ID NO: 1 and a mutation at a position corresponding to position 384 of SEQ ID NO: 1.

18. A polynucleotide comprising a Factor IX nucleotide sequence that encodes a modified Factor IX polypeptide of any one of the preceding claims.

19. The polynucleotide of claim 18, wherein:
(i) the Factor IX nucleotide sequence is at least 80%, at least 90%, at least 95%, at least 98%, or at least 99% identical to a fragment of at least 1200, at least 1350, or at least 1650 nucleotides of SEQ ID NO: 8; and/or
(ii) the Factor IX nucleotide sequence is at least 80%, at least 90%, at least 95%, at least 98%, or at least 99% identical to SEQ ID NO: 8; and/or
(iii) the Factor IX nucleotide sequence comprises a codon that encodes an amino acid at a position corresponding to position 347 of SEQ ID NO: 1, and wherein the codon that encodes an amino acid at a position corresponding to position 347 of SEQ ID NO: 1 encodes arginine; and/or
(iv) the Factor IX nucleotide sequence comprises a codon that encodes an amino acid at a position corresponding to position 347 of SEQ ID NO: 1, and the codon that encodes an amino acid at a position corresponding to position 347 of SEQ ID NO: 1 is CGT, CGC, CGA, CGG, AGA or AGG; and/or
(v) the Factor IX nucleotide sequence comprises a codon that encodes an amino acid at a position corresponding to position 347 of SEQ ID NO: 1, and the codon that encodes an amino acid at a position corresponding to position 347 of SEQ ID NO: 1 is AGG; and/or
(vi) the Factor IX nucleotide sequence comprises a codon that encodes an amino acid at a position corresponding to position 384 of SEQ ID NO: 1, and wherein the codon that encodes an amino acid at a position corresponding to position 384 encodes alanine; and/or
(vii) the Factor IX nucleotide sequence comprises a codon that encodes an amino acid at a position corresponding to position 384 of SEQ ID NO: 1, and the codon that encodes an amino acid at a position corresponding to position 384 of SEQ ID NO: 1 is GCT, GCC, GCA or GCG; and/or
(viii) the Factor IX nucleotide sequence comprises a codon that encodes an amino acid at a position corresponding to position 384 of SEQ ID NO: 1, and the codon that encodes an amino acid at a position corresponding to position 384 of SEQ ID NO: 1 is GCC; and/or
(ix) the Factor IX nucleotide sequence comprises a codon that encodes an amino acid at a position corresponding to position 384 of SEQ ID NO: 1, and wherein the codon that encodes an amino acid at a position corresponding to position 384 encodes leucine; and/or
(x) the Factor IX nucleotide sequence comprises a codon that encodes an amino acid at a position corresponding to position 384 of SEQ ID NO: 1, and the codon that encodes an amino acid at a position corresponding to position 384 of SEQ ID NO: 1 is CTX, wherein X is any nucleotide; and/or
(xi) the Factor IX nucleotide sequence comprises a codon that encodes an amino acid at a position corresponding to position 384 of SEQ ID NO: 1, and the codon that encodes an amino acid at a position corresponding to position 384 of SEQ ID NO: 1 is CTC or CTG; and/or
(xii) the Factor IX nucleotide sequence is operably linked to a transcription regulatory element; and/or
(xiii) the Factor IX nucleotide sequence is operably linked to a transcription regulatory element which comprises a liver-specific promoter and/or an enhancer; and/or
(xiv) the Factor IX nucleotide sequence is operably linked to a transcription regulatory element which is at least 80%, at least 90%, at least 95%, at least 98%, or at least 99% identical to SEQ ID NO: 9.

20. A viral particle comprising a recombinant genome comprising the polynucleotide of any one of claims 18-19.

21. The viral particle of claim 20:
(i) which is an AAV, adenoviral, or lentiviral viral particle; and/or
(ii) which is an AAV viral particle; and/or
(iii) wherein the viral particle further comprises:
a) AAV2 ITRs;
b) a polyA sequence;
c) an origin of replication; and/or
d) two resolvable ITRs; and/or
(iv) wherein administration of the viral particle is associated with a shortened bleeding time in a mouse tail clip assay compared to administration of an equivalent dose of an equivalent viral particle comprising a Factor IX nucleotide sequence encoding a Factor IX polypeptide of SEQ ID NO: 1 or SEQ ID NO: 2.

22. A composition comprising the modified Factor IX polypeptide, polynucleotide or viral particle of any one of the preceding claims and a pharmaceutically acceptable excipient.

23. The modified Factor IX polypeptide, polynucleotide, viral particle or composition of any one of the preceding claims for use in a method of treatment.

24. The modified Factor IX polypeptide, polynucleotide, viral particle or composition for use of claim 23, wherein the method of treatment comprises administering an effective amount of the modified Factor IX polypeptide, polynucleotide, viral particle or composition of any one of claims 1-22 to a patient.

25. The modified Factor IX polypeptide for use, polynucleotide for use, viral particle for use or, composition for use of claim 23 or 24, wherein:
(i) the method of treatment is a method of treating haemophilia; and/or
(ii) the method of treatment is a method of treating is haemophilia B; and/or
(iii) the patient has antibodies or inhibitors to Factor IX.
